(19) United States
(12) Patent Application Publication
(10) Pub. No.: US 2026/0027151 A1
(43) Pub. Date: Jan. 29, 2026

(54) PROCESS FOR REMOVING Pb2+ IONS FROM BODILY FLUIDS USING TITANATE-BASED ION EXCHANGERS

(71) Applicant: UOP LLC, Des Plaines, IL (US)

(72) Inventors: Gregory J. Lewis, Santa Cruz, CA (US); William Christopher Sheets, Glenview, IL (US); Mimoza Sylejmani-Rekaliu, Bensenville, IL (US); Paulina Jakubczak, Elk Grove Village, IL (US); Evgeny T. Kolev, Arlington Heights, IL (US); Subhadra Chudasama, Inverness, IL (US)

(21) Appl. No.: 19/088,180

(22) Filed: Mar. 24, 2025

Related U.S. Application Data
(60) Provisional application No. 63/674,401, filed on Jul. 23, 2024.

(51) Int. Cl. A61K 33/24; A61K 9/00; C01G 23/047

(52) U.S. Cl. CPC A61K 33/24; A61K 9/0053; C01G 23/047; C01P 2002/02; C01P 2004/32; C01P 2004/52; C01P 2004/61

(57) ABSTRACT

A process for removing Pb²⁺ ions from fluids is disclosed. More particularly, an intracorporeal process for removing Pb²⁺ ions from gastrointestinal fluids is disclosed. The process involves contacting gastrointestinal fluid with a titanate-based ion exchanger represented by the empirical formula:

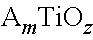

where A is sodium, potassium, lithium, magnesium, calcium, hydronium, or mixtures thereof. The alkali titanate ion exchanger is synthesized from specific Ti reagents, including nano-sized TiO₂ and preformed spray dried TiO₂ spheres, that impart properties such as favorable particle size and particle size distributions that are beneficial for treating the body.

### RELATED APPLICATIONS

This application claims priority to U.S. Provisional Patent Application Ser. No. 63/674,401, filed on Jul. 23, 2024, the entirety of which is incorporated herein by reference.

### FIELD OF THE INVENTION

This invention relates to intracorporeal processes for removing Pb²⁺ ions from bodily fluids, especially gastrointestinal fluids, using alkali titanate-based ion exchangers. The gastrointestinal fluid is contacted directly with an alkali titanate-based ion exchange composition, which can remove the Pb²⁺ toxin. The titanate-based ion exchangers used in this process are synthesized from nano-sized titanium dioxide or preformed spray dried titanium dioxide spheres resulting in favorable particle size and particle size distribution properties that are beneficial for treating the body.

### BACKGROUND

Lead has become widely distributed in the biosphere only in the past few thousand years, entirely as the result of human activity. Once introduced into the environment, lead persists. Investigations of human skeletal remains indicate that the body lead burden of today's populations is 500-1000 times greater than that of their pre-industrial counterparts (See NE J. Med., vol. 326, no. 19, pp. 1293-1294, 1992). By far the largest contributor to global environmental lead contamination was the use of lead in gasoline, mainly between 1965 and 1990 (See Environ. Health Perspect., vol. 110, no. 7, pp. 721-728, 2002). With lead now removed from gasoline across the developed world, blood lead levels (BLLs) in the US and worldwide are expected to continue their slow decline (See Am. J. Med., vol. 129, pp. 1213-1218, 2016). However, hot spots from smelting, mining, aging houses, water lines, highways, and metal recycling operations—some of them ongoing and others the legacy of the past—remain significant problems. Despite a century of accumulated evidence about its danger to the health of children, lead too often continues to be added to paints, pigments, toys, traditional medications, cosmetics, and other consumer products, especially as manufacturing shifts to developing countries that lack environmental and product content controls and policies. Dust from lead-based paint remains a significant source of exposure (See J. Pediatrics, vol. 140, no. 1, pp. 40-47, 2002). Soils in older areas of cities are often highly contaminated by lead, owing to past use of lead additives in gasoline and industrial sources. Soils are not passive sources and periodic re-suspension of fine lead-contaminated soil dust particulates create seasonal variations of lead exposure for urban dwellers (See Atmos. Environ., vol. 49, pp. 302-310, 2012). Furthermore, there exists a coupling between inhaled and ingested lead. Inhalation studies examining lead particles (˜1 μm) deposited in the back of the nose have demonstrated that they are typically swallowed over time. In addition, particles deposited in the tracheobronchial region may be cleared from the lungs via the mucociliary escalator, reaching the gastrointestinal system via the esophagus (See Radiat. Prot. Dosim., vol. 127, pp. 31-34, 2007). A change in water supply utilizing old, contaminated infrastructure brought lead-contaminated water to Flint, Michigan where significant elevation of blood lead levels (BLLs) were observed in children (See Am. J. Public Health, vol. 106, no. 2, pp. 283-290, 2016). A report released by the Natural Resources Defense Council in 2018 details how many other communities around the country are failing to adequately ensure that their water supplies remain free of lead (See E. Olson and K. P. Fedinick, Natural Resources Defense Council, New York, NY, 2016). Experts have estimated that 6 to 10 million lead service lines are being used in the US, serving 15 to 22 million Americans, most of which were installed at least 50 years ago (See J. Am. Water Works Assoc., vol. 108, no. 4, pp. E182-E191, 2016).

Lead is highly toxic, it can harm the brain, kidneys, bone marrow and other bodily systems, especially those of young children. The Third National Health and Nutritional Examination (NHANES III, Phase 2, 1991-1994) found that 4.4% of children under 6 years old in the United States had BLLs above 10 μg/dL, a level that is considered “poisoned.” (See Eliminating Childhood Lead Poisoning: A Federal Strategy Targeting Lead Paint Hazards; President's Task Force on Environmental Health Risks and Safety Risks to Children, February, 2000). Exposure via lead-containing paint is the main culprit, with the impact greater on children from low-income and minority families living in older housing, as 16% of these children are poisoned compared to the national average of 4.4% for all children (See Morbidity and Mortality Weekly Report, U.S. Department of Health and Human Services/Public Health Service, Vol 46, No. 7, Feb. 21, 1997, p. 141-146). As BLLs increase, the variety and morbidity of lead poisoning increases, including reduced IQ, decreased hearing, and decreased growth. Behavior problems in children can be observed at BLL=10 μg/dL, impaired nerve function at 20 μg/dL, reduced vitamin C metabolism at 30 μg/dL, damage to hematopoiesis at 40 μg/dL, severe stomach cramps above 50 μg/dL, and severe brain and kidney damage and severe anemia between 50-100 μg/dL. Even low-level elevations in children's blood lead concentrations, at concentrations below 5 μg/dL, can result in decrements in cognitive functions, as measured by IQ scores and academic performance (See Public Health Rep., vol. 115, pp. 521-529, 2000; and Environ. Health Perspect., vol. 113, pp. 894-899, 2005). For a given level of exposure, lead-associated IQ losses are proportionately greater at the lowest blood lead concentrations. The IQ decrease associated with an increase in blood lead concentration from <1 to 30 μg/dL was 9.2 IQ points, but the decrease associated with an increase in blood lead concentration from <1 to 10 μg/dL was 6.2 IQ points. The population impact of lead on intellectual abilities is substantial. Despite the dramatic reductions in blood lead levels, lead toxicity accounts for an estimated total loss of 23 million IQ points among a 6-year cohort of contemporary US children (See Environ. Health Perspect., vol. 120, pp. 501-507, 2012). There is an inverse relationship between early childhood exposure to lead and performance on tests of cognitive function and behavior 10 and 20 years after the blood lead levels were measured (See Pediatrics, vol. 90, pp. 855-861, 1992). Early exposures have also been linked to increased rates of hyperactivity, inattentiveness, failure to graduate from high school, conduct disorder, juvenile delinquency, drug use and incarceration (See PLos Medicine, vol. 5, p. e101, 2008). With respect to kidney damage, a study that examined the NHANES III (1988-1994) reported that among 769 adolescents with a median blood lead concentration of 1.5 μg/dL (15 ppb), a doubling of the concentration led to a significant reduction in the glomerular filtration rate (See Arch Intern Med., vol. 170, pp. 75-82, 2010). Numerous experimental and many epidemiology studies suggest that lead is a risk factor for cardiovascular disease (CVD) and mortality. While the definition of elevated BLL in adults is 5 μg/dL, a recent study concluded that low-level environmental lead exposure (i.e., <5 μg/dL) is an important risk factor for CVD mortality (See Lancet Public Health, vol. 3, pp. e177-184, 2018). The authors tracked NHANES III (1988-1994) participants through 2011 (˜14,000 adults) and examined the relationship between lower levels of lead, down to 1 μg/dL, and CVD and ischemic heart disease mortality. Analysis of the data suggested that ˜400,000 US deaths annually are attributable to lead exposure, highlighting the potential risk factor of adult BLL concentrations of 1-5 g/dL.

Chelation therapy has been used to remove lead from the blood. An optimal chelating drug should increase lead excretion, be administered easily, be affordable and safe. However, lead-chelate complexes may persist in tissues where the binding occurred or be redistributed to other tissues. Increased symptoms commonly reported with aggressive initiation of chelation therapies are cited as a contraindication to any use of chelators. Chelating agents effectively remove lead in the blood and are indicated for acutely affected patients with a BLL>45 μg/dL, administered in consultation with a specialist (See Environmental Health Perspectives, vol. 115, no. 3, pp. 463-471, 2007). Chelating agents used to treat lead poisoning include intravenous (IV) calcium disodium edetate (CaNa₂EDTA), intramuscular dimercaprol and oral 2,3-dimercaptosuccinic acid (DMSA, also known as succimer).

EDTA, ethylenediaminetetraacetic acid, is used in the CaNa₂EDTA form to avoid hypocalcemia and possibly death due to its capability to bind calcium. It can also bind other bioavailable cations such as zinc, copper, and iron (See Int. J. Environ. Res. Public Health, vol. 7, pp. 2745-2788, 2010). It is often used in the treatment of severe lead poisoning and is administered intravenously in a hospital setting because substantial monitoring is required including renal function, cardiac activity, and daily phlebotomy to monitor serum electrolytes. Pain and swelling may result from the parenteral administration of CaNa₂EDTA, while other adverse effects include fever, nausea, vomiting, and nephrotoxicity, such as microscopic hematuria and proteinuria. CaNa₂EDTA has been reported to spread lead to other tissues and can increase lead concentrations in the central nervous system and may cause encephalopathy. After a single dose of CaNa₂EDTA, urinary lead levels increase, blood levels decrease, and brain levels increase significantly due to redistribution of lead into the brain.

DMSA is indicated for BLL>45 μg/dL, for treatment of acute lead poisoning situations and does not require administration in a hospital. Oral DMSA is dosed typically at 30 mg/kg/day for 5 days (See Med. Toxicol. Adverse Drug Exp., vol. 3, pp. 499-504, 1988), often followed by a 14-day course of 20 mg/kg/day, a treatment regimen that increases urine lead excretion and reduces blood lead concentrations significantly. Drug-induced neutropenia can occur, weekly blood count is recommended, and treatment should be discontinued if neutrophil counts get too low. Adverse effects of DMSA treatment include nausea, vomiting, diarrhea, loose stool, metallic taste that are experienced singly or together by 12% of children and 21% of adults (See U.S. Pat. No. 11,083,748). Back pain, abdominal cramps, chills, and flu-like symptoms have also been reported in 5% of children and 16% of adults.

Dimercaprol, also known as British Anti-Lewisite (BAL) was originally developed as an experimental antidote to Lewisite, an arsenic-based poison gas. To administer, it is dissolved in peanut oil and injected into the muscles, a procedure not well tolerated by children. The small molecule can enter the blood-brain barrier and can be used to treat encephalopathy, often in combination with CaNa₂EDTA. The most common side effect of treatment with dimercaprol is a 50 mmHg rise in systolic and diastolic blood pressure among many others. In the 1960's, dimercaprol was modified into DMSA, which has fewer side effects and thus has fallen out of favor for treatment of lead poisoning.

Chelation therapy with CaNa₂EDTA, DMSA, and dimercaprol all are effective in reducing blood lead levels. However, it is well known that after these treatments there is often a rebound in the BLL, which likely results from absorbed lead emanating from bone and soft tissues. Considering the considerable side effects associated with chelation therapy, it is not practiced when addressing the main health problems associated with low level lead poisoning discussed above.

Zeolites, microporous aluminosilicate ion exchangers based on tetrahedral frameworks, have been proposed for treating chronic lead poisoning and are taken in pill form (See U.S. Pat. No. 11,038,748). However, zeolites have limited stability in blood and especially the acidic environment of the gastrointestinal tract. In acidic solution, Al naturally has octahedral coordination and can be extracted from the tetrahedral zeolite framework. Indeed, treatment with acidic solution is one strategy to modify zeolite composition via dealumination, see U.S. Pat. No. 6,982,074. In U.S. Pat. No. 11,038,748, a particulate sodium aluminosilicate is claimed with 90% of the particulates between a size of 90 and 150 μm. A process is disclosed to achieve this particle size distribution that seems to involve screening using sieves. More recently, examples of microporous ion exchangers that are essentially insoluble in fluids, such as bodily fluids (especially blood), have been developed, namely the zirconium-based silicates and titanium-based silicates of U.S. Pat. Nos. 5,888,472; 5,891,417; and 6,579,460. The use of these zirconium-based silicate or titanium-based silicate microporous ion exchangers to remove toxic ammonium cations from blood or dialysate is described in U.S. Pat. Nos. 6,814,871; 6,099,737; and 6,332,985. Additionally, it was found that some of these compositions were selective in potassium ion exchange and could remove potassium ions from bodily fluids to treat the disease hyperkalemia, which is discussed in U.S. Pat. Nos. 8,802,152; 8,808,750; 8,877,255; 9,457,050; 9,662,352; 9,707,255; 9,844,567; 9,861,658; 10,413,569; 10,398,730; US 2016/0038538; US 2016/0271174; and U.S. Pat. No. 10,695,365. Ex-vivo applications of these materials, for instance in dialysis, are described in U.S. Pat. No. 9,943,637. One of these patents, U.S. Pat. No. 8,802,152, deals with the use of zirconium silicate compositions to treat hyperkalemia via the gastrointestinal tract and identifies particles smaller than 3 microns as undesirable, as these may be absorbed into the patient's bloodstream causing adverse effects, including accumulation in the kidneys. Screening techniques are used to reduce or nearly eliminate particles less than 3 microns from the zirconium silicate product.

Many ion-exchangers have been developed for removal of “heavy metals” from various streams, often waste streams. The heavy metals often include lead, mercury, cadmium, zinc, iron, chromium, copper, cobalt, nickel and even arsenic. In many applications, these metals are lumped together as if they have the same ion-exchange properties, but in fact there is great variation. Zeolites are well known ion-exchangers and the ability of a particular zeolite to remove Pb²⁺ from solution does not mean that it will remove Hg²⁺ from solution. In U.S. Pat. No. 9,233,856, which is incorporated by reference, it is shown that the uptake of Hg²⁺ by zeolites is highly dependent on the framework charge density or equivalently, the Si/Al ratio. High charge density Si/Al=1 zeolites like X (FAU topology) and 4A (LTA topology) are shown to have almost no affinity for Hg²⁺ under the test conditions while they easily remove Ca²⁺ and Mg²⁺ (See U.S. Pat. No. 9,233,856, Example 10). In the same test using UZM-9, a zeolite with Si/Al=5.50 that has the same LTA zeolite topology as zeolite 4A, the situation is reversed and there is high selectivity for Hg²⁺, while the selectivity to Ca²⁺ and Mg²⁺ is highly diminished. In the case of zeolite 4A and UZM-9, this result decouples structure from the framework charge density and shows the importance of the latter in ion-exchange selectivity (See U.S. Pat. No. 9,233,856, Example 11). Meanwhile, our investigations show that zeolite X is excellent at removing Pb²⁺ from aqueous solution, while it performs poorly at removing Hg²⁺ (See Comparative Example 13 in U.S. Pat. No. 9,233,856). Hence, one cannot generalize that if an ion exchanger is selective for one metal cation that it will perform similarly for another metal cation, the data must be collected for each remediated metal cation/resident metal cation/ion exchanger combination.

A process was disclosed for the removal of Sr²⁺ ions from bodily fluids using Zr, Ti, Sn-based metallate ion exchangers, see U.S. Pat. No. 11,577,014. The majority of these are crystalline and amorphous metallosilicates, while a metal oxide was also disclosed, a commercial sodium nonatitanate sample. A process was also disclosed for the removal of Hg²⁺ from bodily fluids utilizing Ti metallates, see U.S. Pat. No. 11,484,875. Ti was required in the compositions with Nb and Si optional. Very specific topology/composition combinations were required for efficient Hg²⁺ removal, including metal silicates Ti—Nb sitinakite and acid treated zorite as well as a metal oxide, a commercial sodium nonatitanate from Allied-Signal. Another recent US patent application, U.S. Pat. No. 11,964,266 discloses the use of metallosilicates and metal oxides for the removal of cobalt, lead, cadmium, and chromium ions from bodily fluids using Zr, Ti, Sn-based metallate ion exchangers. The majority of these ion exchangers are crystalline and amorphous metallosilicates while two metal oxides were disclosed, a commercial sample of sodium nonatitanate from Allied-Signal and a commercial potassium octatitanate product from Honeywell. Unlike the sodium nonatitanate, the potassium octatitanate was not effective for efficient Hg²⁺ and Sr²⁺ removal. The test for Pb²⁺ uptake employed in U.S. Pat. No. 11,964,266 had a rather high detection limit of 200 ppb, which limited the ability to differentiate the performance of the various compositions. While these applications claimed forming of the ion-exchangers into various shapes, there was no consideration given to size of the as-synthesized crystallites and particulates.

### SUMMARY OF THE INVENTION

The present disclosure provides for, and includes, a process to remove Pb²⁺ ions from bodily fluids, which uses polycrystalline aggregate titanate-based ion exchangers that are essentially insoluble in bodily fluids, especially gastrointestinal fluids. The titanate-based ion exchangers have an empirical formula on an anhydrous basis of:

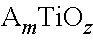

where A is an exchangeable cation selected from the group consisting of potassium ion, sodium ion, lithium ion, calcium ion, magnesium ion, hydronium ion or mixtures thereof, “m” is the mole ratio of A to Ti and has a value from 0.05 to 0.60, and “z” is the mole ratio of O to Ti and has a value from 2.05 to about 2.60 and are synthesized from specific titania reagents, including nano-sized titania powder, preformed spray dried titania spheres, or both. Since these compositions are essentially insoluble in bodily fluids (at neutral and mildly acidic or basic pH) and exhibit particle sizes sufficiently large to avoid absorption in the gastrointestinal tract, they can be orally ingested to remove Pb²⁺ toxins in the gastrointestinal system.

As stated, this invention relates to an intracorporeal process for removing Pb²⁺-containing toxins from gastrointestinal fluids, the process comprising contacting the fluid containing the toxins with a polycrystalline aggregate titanate-based ion exchanger resulting in an ion exchanged ion exchanger thereby removing the toxins from the fluid, the titanate-based ion exchanger having an empirical formula on an anhydrous basis of:

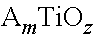

where A is an exchangeable cation selected from the group consisting of potassium ion, sodium ion, lithium ion, calcium ion, magnesium ion, hydronium ion or mixtures thereof, “m” is the mole ratio of A to Ti and has a value from 0.05 to 0.60, “z” is the mole ratio of O to Ti and has a value from 2.05 to 2.60 where the titanate-based ion exchanger is synthesized from specific titania reagents, including nano-sized titania powder, preformed spray dried titania spheres, or both.

Another embodiment of the invention is the synthesis of the titanate-based ion exchange composition from nano-sized TiO₂ powders in a highly alkaline reaction mixture, imparting unique properties to the resulting titanate-based ion exchanger. Utilizing nano-sized titania powder enables the formation of large macroporous polycrystalline aggregate particles from the hydrothermal conversion in highly alkaline reaction mixture, the product particles exhibiting a large enough size to avoid absorption in the gastrointestinal tract and a more uniform particle size distribution than obtained with other titania powders.

Another embodiment of the invention is the hydrothermal synthesis of the titanate-based ion exchange composition from pre-formed TiO₂, e.g., from spray dried TiO₂ spheres in highly alkaline reaction mixtures. The resulting alkali titanate spheres that are large enough to avoid absorption into the gastrointestinal tract. In this case, the source of titania powder used to produce the TiO₂ spheres may be nano-sized titania or other titania powders that are not nano-sized.

In yet another embodiment of the invention is provided a process for removing Pb²⁺ containing toxins from fluids, the process comprising contacting the fluid containing the toxins with a titanate-based ion exchanger resulting in an ion exchanged ion exchanger thereby removing the toxins from the fluid, the titanate-based ion exchanger having an empirical formula on an anhydrous basis of:

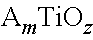

where A is an exchangeable cation selected from the group consisting of potassium ion, sodium ion, lithium ion, calcium ion, magnesium ion, hydronium ion or mixtures thereof, “m” is the mole ratio of A to Ti and has a value from 0.05 to 0.60, “z” is the mole ratio of O to Ti and has a value from 2.05 to 2.60 wherein the titanate-based ion exchanger is synthesized from specific titania reagents, including nano-sized titania powder, preformed spray dried titania spheres, or both.

The present disclosure also provides for, and includes, a method for manufacturing a tablet or capsule or for oral administration, the tablet or capsule comprising an alkali titanate ion exchanger having an empirical formula on an anhydrous basis of:

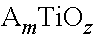

wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.05 to 0.60, and “z” is the mole ratio of O to Ti and has a value from 2.05 to 2.60, wherein the titanate-based ion exchanger is synthesized from specific titania reagents, including nano-sized titania powder, preformed spray dried titania spheres, or both and the alkali titanate ion exchanger exhibits a median particle size of greater than 3 microns (μm), the method comprising:
   (a) forming a reaction mixture comprising reactive sources of A, Ti, and water;
   (b) heating the reaction mixture for a period of time to form a titanate ion exchanger;
   (c) treating the synthesized alkali titanate ion exchanger via acid extraction and/or ion exchange with alkali metals, alkaline earth metals, or mixtures thereof, to form the alkali titanate ion exchanger with the desired composition;
   (d) optionally admix the alkali titanate ion exchanger with one or more pharmaceutically acceptable adjuvants, diluents or carriers to form an alkali titanate ion exchanger medicament;
   (e) forming a capsule or tablet comprising the alkali titanate ion exchanger medicament;
      wherein the reaction mixture comprises a composition expressed in terms of mole ratios of the oxides of:

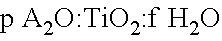

wherein “p” has a value from about 4 to 40 and “f” has a value from 20 to 1000.

These and other embodiments will become clearer after a detailed description of the invention.

### BRIEF DESCRIPTION OF THE FIGURES

FIG. 1 shows a scanning electron microscope image (SEM) of nano-titania powder used in Example 1 synthesis.

FIG. 2 shows an SEM image of the Example 1 alkali titanate product resulting from the quiescent digestion of nano-titania in 8.5 M KOH solution at 225° C. for 24 hr.

FIG. 3 shows an SEM image of the Example 2 alkali titanate product resulting from the tumbled digestion of preformed TiO₂ spheres in 8.5 M KOH solution at 225° C. for one day.

FIG. 4 shows the particle size distribution of the sodium nonatitanate prior art material from comparative Example C1.

FIG. 5 shows the particle size distribution of the potassium octatitanate prior art material from comparative Example C2.

FIG. 6 shows the particle size distribution of Example C3 product prepared from anatase/rutile TiO₂ powder source digested quiescently in 8.5 M KOH solution at 175° C. for 24 hr.

FIG. 7 shows the particle size distribution of Example C4 product prepared from anatase TiO₂ powder source digested quiescently in 7.5 M NaOH solution at 200° C. for 24 hr.

FIG. 8 shows the particle size distribution of Example C5 product prepared from anatase TiO₂ powder source digested quiescently in 8.5 M KOH solution at 200° C. for 24 hr.

FIG. 9 shows the particle size distribution of Example 1 product prepared from nano-sized anatase TiO₂ powder source digested quiescently in 8.5 M KOH solution at 200° C. for 24 hr.

FIG. 10 shows the particle size distribution of Example 2 product prepared from rutile/anatase TiO₂ powder source digested with tumbling at 30 rpm in 8.5 M KOH solution at 225° C. for one day.

### DETAILED DESCRIPTION

### A. Alkali Titanate Ion Exchangers

As stated, applicants have developed anew process for removing Pb²⁺-containing toxins from gastrointestinal fluids. One essential element of the instant process is an ion exchanger which has a large capacity and strong affinity, i.e., selectivity for Pb²⁺ ions, both free and complexed. Another essential element of the instant process is that the titanate-based ion exchangers are synthesized from specific titania reagents, including nano-sized titania powder, preformed spray dried titania spheres, or both, which impart favorable properties such as a median particle size of greater than 3 microns (μm), which helps avoid undesirable absorption during treatment in the gastrointestinal tract. These titanate-based compositions are identified as alkali titanate-based compositions as synthesized and may additionally be modified by ion exchange. They are further identified by their composite empirical formula (on an anhydrous basis) which is:

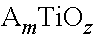

The composition has a framework structure(s) composed of at least TiO_{6/n} octahedral units where n may be 2 or 3 or both, although other coordination environments may occur for Ti. A is an exchangeable cation selected from the group consisting of potassium ion, sodium ion, lithium ion, calcium ion, magnesium ion, hydronium ion or mixtures thereof, “m” is the mole ratio of A to Ti and has a value from 0.05 to 0.60, and “z” is the mole ratio of O to Ti and has a value from 2.05 to 2.60.

The titanate-based compositions of this invention are prepared by a hydrothermal crystallization of a reaction mixture prepared by combining a reactive source of titanium, at least one alkali metal, and water. The alkali metal acts as a framework charge balancing agent as well as a templating agent. When the Ti source is TiO₂ powder, examples of titanium metal sources include, but are not limited to nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof, where nano-sized is defined as a particle size of 200 nm or less, preferably 100 nm or less. When the Ti source is preformed TiO₂ spheres, examples of Ti metal sources include amorphous TiO₂, amorphous titanium oxyhydroxide, anatase titanium dioxide, rutile titanium dioxide, brookite titanium dioxide, nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof. Examples of A alkali sources include potassium hydroxide, sodium hydroxide, lithium hydroxide, sodium halide, potassium halide, lithium halide, sodium acetate, potassium acetate, and lithium acetate. Generally, the hydrothermal process used to prepare the alkali titanate ion exchange compositions of this invention involves forming a reaction mixture which in terms of molar ratios of the oxides is expressed by the formula:

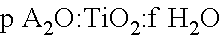

where “p” has a value from about 4 to 40, and “f” has a value from 20 to 1000. The reaction mixture is prepared by mixing the desired sources of alkali metal, titanium dioxide, and water to give the desired mixture. It is also necessary that the final reaction mixture have a basic pH and preferably a pH of at least 13. The basicity of the mixture is controlled by adding alkali hydroxide. Having formed the reaction mixture, it is next reacted at a temperature of about 85° C. to about 225° C. for a period of about 0.5 to about 30 days in a sealed reaction vessel under autogenous pressure. After the allotted time, the mixture is filtered or centrifuged to isolate the solid product which is washed with deionized water and dried in air. As stated, the compositions of this invention have a framework structure composed mostly of octahedral TiO_{6/n} units, but other coordination environments may be present. The titanate-based ion exchanger of his invention may exhibit various levels of crystallinity and may be amorphous.

As synthesized, the compositions of this invention will contain some of the alkali metal templating agent in the pores, between layers and chains, or in other charge balancing positions. These metals are described as exchangeable cations, meaning that they can be exchanged with other (secondary) A′ cations. Generally, the A exchangeable cations can be exchanged with A′ cations selected from other alkali metal cations (K⁺, Na⁺, Li⁺), alkaline earth cations (Mg²⁺, Ca²⁺), hydronium ion or mixtures thereof. It is understood that the A′ cation is different from the A cation. The methods used to exchange one cation for another are well known in the art and involve contacting the compositions with a solution containing the desired cation (at molar excess) at exchange conditions. Exchange conditions include a temperature of about 25° C. to about 100° C. and a time of about 20 minutes to about 2 hours. The particular cation (or mixture thereof), which is present in the final product will depend on the particular use of the composition and the specific composition being used. For instance, for the treatment of a hypocalcemic patient low on Ca²⁺, one specific composition is a Ca²⁺-containing ion exchanger where the A′ cation is a mixture of Na⁺, Ca²⁺ and H⁺ ions.

In an aspect, synthesized powder forms of the alkali titanate ion exchangers of the present disclosure are generally particles that are greater than 3 microns in size, which is desirable since particles that are less than 3 microns in size can be absorbed by the body. However, it may be desirable to have a subset of these particles being smaller than 3 microns with an upper limit of 3% less than 3 microns in size by volume. In an aspect, an alkali titanate ion exchanger of the present disclosure exhibits a median particle size greater than 3 microns. In an aspect, an alkali titanate ion exchanger of the present disclosure has a median particle size ranging from 25 to 125 microns.

It is also within the scope of the invention that these ion exchange compositions can be used in as-synthesized powder form or can be formed into various shapes by means well known in the art. Examples of these various shapes include pills, extrudates, spheres, pellets, and irregularly shaped particles. This has previously been demonstrated in U.S. Pat. No. 6,579,460B1 and U.S. Pat. No. 6,814,871B1. The forming process may include the addition of a binding agent, for example, zirconia, that may require annealing, which may occur by calcination in air at temperatures up to 350° C. for 2 to 6 hours. The forming may also occur at several scales, for instance at one scale for the synthesis of the titanate-based ion exchanger and at another scale for delivery of the titanate-based ion exchanger to the body. For instance, in Example 3, spray dried TiO₂ spheres are used as the starting material for the synthesis of the titanate-based ion exchangers of tens of microns in diameter, which can then be formed into a larger pill for delivery to the body. The formed pills or other shapes of the titanate-based ion exchangers can be ingested orally and pick up toxins in the gastrointestinal fluid as the ion exchanger passes through the intestines and is finally excreted. It is possible to protect the ion exchangers from the high acid content in the stomach, the shaped articles may be coated with various coatings which will not dissolve in the stomach, but dissolve in the intestines.

As provided herein, in an aspect, the titanate ion exchanger compositions of the present disclosure can have particular utility in adsorbing Pb²⁺-containing toxins, including free Pb²⁺ ions and Pb²⁺ from complexed Pb²⁺ ions, from gastrointestinal fluids. The titanate-based ion exchangers of this invention are not limited to the treatment of gastrointestinal fluids but may also be used to remove Pb²⁺ from other bodily fluids, such as blood or dialysate solutions. As used herein and in the claims, while the current focus is gastrointestinal fluids, bodily fluids will include but not be limited to blood, blood plasma, and gastrointestinal fluids. Also, the compositions are meant to be used to treat bodily fluids of any mammalian body, including but not limited to humans, cows, pigs, sheep, monkeys, gorillas, horses, dogs, etc. The instant process is particularly suited for removing toxins from a human body.

As provided herein, in an aspect, while the alkali titanate ion exchanger compositions of the present disclosure may be synthesized with a variety of exchangeable cations (“A”), it may be preferable to exchange the cation with secondary cations (A′) which are more compatible with blood or do not adversely affect the blood. Without being limited by theory, compositional requirements for the ion exchanger will vary with the needs of the patient. For this reason, preferred cations are potassium, sodium, lithium, calcium, hydronium, and magnesium. In an aspect, preferred compositions include those containing potassium ions. In another aspect, preferred compositions include those containing hydronium ions. In another aspect, preferred compositions include those containing a combination of potassium and hydronium ions. The relative amounts of any two cations within an alkali titanate ion exchanger can vary considerably, and may depend on the composition itself as well as the concentration of the two ions in the blood.

In an aspect, the alkali titanate ion exchanger according to the present disclosure is in a solid dosage form. Solid dosage forms used for oral administration may include capsules, tablets, pills, powders, extrudates, spheres, pellets, granules, and irregularly shaped particles. Among these solid dosage forms, an active alkali titanate ion exchanger is mixed with at least one conventional inert excipient (or vehicle), such as sodium citrate or dicalcium phosphate, or mixed with any one or more of the following ingredients: (a) a filler or a compatibilizer, such as starch, lactose, sucrose, glucose, mannitol, and silicic acid; (b) a bonding agent, such as hydroxymethyl cellulose, alginate, gelatin, polyvinylpyrrolidone, sucrose, and gum arabic; (c) a moisturizer, such as glycerin; (d) a disintegrant, such as agar, calcium carbonate, potato starch or tapioca starch, alginic acid, some composite silicates, and sodium carbonate; (e) a slow solvent, such as paraffin; (f) an absorbing accelerator, such as quaternary amine compounds; (g) a wetting agent, such as cetyl alcohol and glyceryl monostearate; (h) an adsorbent, such as kaolin; and (i) a lubricant, such as talc, calcium stearate, magnesium stearate, solid polyethylene glycol, sodium dodecyl sulfate, or a mixture thereof. Dosage forms of capsules, tablets, and pills may also contain a buffer agent.

In an aspect, solid dosage forms such as tablets, sugared pills, capsules, pills, and granules may be prepared using coatings and shells, such as enteric coatings and other materials known in the art. The solid dosage forms may contain opacifiers, and moreover, active alkali titanate ion exchangers or alkali titanate ion exchangers in such compositions may be released in a portion of the digestive tract in a delayed manner. Non-limiting examples of embedding components that can be employed are polymeric materials and waxy materials. The active alkali titanate ion exchangers may also be formed into microcapsules with one or more of the above excipients.

In an aspect, an alkali titanate ion exchanger according to the present disclosure is in tablet form. In an aspect, an alkali titanate ion exchanger according to the present disclosure is in capsule form. In an aspect, an alkali titanate ion exchanger in tablet form further comprises a tablet coating. In an aspect, an alkali titanate ion exchanger in capsule form further comprises a capsule coating.

In an aspect an alkali titanate ion exchanger according to the present disclosure may be admixed with one or more pharmaceutically acceptable adjuvants, diluents or carriers, for example, lactose, saccharose, sorbitol, mannitol; starch, for example, potato starch, corn starch or amylopectin; cellulose derivative; binder, for example, gelatin or polyvinylpyrrolidone; disintegrant, for example cellulose derivative, and/or lubricant, for example, magnesium stearate, calcium stearate, polyethylene glycol, wax, paraffin, and the like, and then compressed into tablets. If coated tablets are required, the cores, prepared as described above, may be coated with a suitable polymer dissolved or dispersed in water or readily volatile organic solvent(s). Alternatively, the tablet may be coated with a concentrated sugar solution which may contain, for example, gum arabic, gelatin, talcum and titanium dioxide.

In an aspect, a sustained-release preparation of an alkali titanate ion exchanger of the present disclosure is administered. Examples of sustained-release preparations include semi-permeable matrices of solid hydrophobic polymers containing the alkali titanate ion exchanger, where the matrices are in the form of shaped articles, e.g., films or microcapsules. Examples of sustained-release matrices include polyesters, hydrogels, and polylactides.

In an aspect, an alkali titanate ion exchanger of the present disclosure has a median particle size in the range of 25 μm to 125 μm. In an aspect, the alkali titanate ion exchanger has a median particle size in the range of 25 μm to 100 μm. In an aspect, the alkali titanate ion exchanger has a median particle size in the range of 25 μm to 75 μm. In an aspect, the alkali titanate ion exchanger has a median particle size in the range of 25 μm to 50 μm. In an aspect, the alkali titanate ion exchanger has a median particle size in the range of 25 μm to 35 μm. In an aspect, the alkali titanate ion exchanger has a median particle size in the range of 35 μm to 100 μm. In an aspect, the alkali titanate ion exchanger has a median particle size in the range of 50 μm to 75 μm. In an aspect, the alkali titanate ion exchanger has a median particle size in the range of 35 μm to 125 μm. In an aspect, the alkali titanate ion exchanger has a median particle size in the range of 50 μm to 125 μm. In an aspect, the alkali titanate ion exchanger has a median particle size in the range of 75 μm to 125 μm. In an aspect, the alkali titanate ion exchanger has a median particle size in the range of 100 μm to 125 μm.

In an aspect, the alkali titanate ion exchanger of the present disclosure has a particle size distribution d₁₀ value of between about 10 microns (μm) and about 80 μm. In an aspect, the alkali titanate ion exchanger has a particle size distribution d₁₀ value of between about 10 μm and about 60 μm. In an aspect, the alkali titanate ion exchanger has a particle size distribution d₁₀ value of between about 10 μm and about 50 μm. In an aspect, the alkali titanate ion exchanger has a particle size distribution d₁₀ value of between about 10 μm and about 40 μm. In an aspect, the alkali titanate ion exchanger has a particle size distribution d₁₀ value of between about 10 μm and about 30 μm. In an aspect, the alkali titanate ion exchanger has a particle size distribution d₁₀ value of between about 10 μm and about 20 μm. In an aspect, the alkali titanate ion exchanger has a particle size distribution d₁₀ value of between about 20 μm and about 80 μm. In an aspect, the alkali titanate ion exchanger has a particle size distribution d₁₀ value of between about 30 μm and about 80 μm. In an aspect, the alkali titanate ion exchanger has a particle size distribution d₁₀ value of between about 40 μm and about 80 μm. In an aspect, the alkali titanate ion exchanger has a particle size distribution d₁₀ value of between about 50 μm and about 80 μm. In an aspect, the alkali titanate ion exchanger has a particle size distribution d₁₀ value of between about 15 μm and about 35 μm. In an aspect, the alkali titanate ion exchanger has a particle size distribution d₁₀ value of between about 20 μm and about 30 μm. In an aspect, the alkali titanate ion exchanger has a particle size distribution d₁₀ value of about 24 μm. In an aspect, the alkali titanate ion exchanger has a particle size distribution d₁₀ value of between about 50 μm and about 70 μm. In an aspect, the alkali titanate ion exchanger has a particle size distribution d₁₀ value of between about 59 μm and about 66 μm. In an aspect, the alkali titanate ion exchanger has a particle size distribution d₁₀ value of about 62 μm.

In an aspect, the alkali titanate ion exchanger of the present disclosure has a particle size distribution d₅₀ value of between about 45 microns (μm) and about 125 μm. In an aspect, the alkali titanate ion exchanger has a particle size distribution d₅₀ value of between about 45 μm and about 110 μm. In an aspect, the alkali titanate ion exchanger has a particle size distribution d₅₀ value of between about 45 μm and about 100 μm. In an aspect, the alkali titanate ion exchanger has a particle size distribution d₅₀ value of between about 45 μm and about 90 μm. In an aspect, the alkali titanate ion exchanger has a particle size distribution d₅₀ value of between about 45 μm and about 80 μm. In an aspect, the alkali titanate ion exchanger has a particle size distribution d₅₀ value of between about 45 μm and about 70 μm. In an aspect, the alkali titanate ion exchanger has a particle size distribution d₅₀ value of between about 45 μm and about 60 μm. In an aspect, the alkali titanate ion exchanger has a particle size distribution d₅₀ value of between about 50 μm and about 125 μm. In an aspect, the alkali titanate ion exchanger has a particle size distribution d₅₀ value of between about 60 μm and about 125 μm. In an aspect, the alkali titanate ion exchanger has a particle size distribution d₅₀ value of between about 70 μm and about 125 μm. In an aspect, the alkali titanate ion exchanger has a particle size distribution d₅₀ value of between about 80 μm and about 125 μm. In an aspect, the alkali titanate ion exchanger has a particle size distribution d₅₀ value of between about 90 μm and about 125 μm. In an aspect, the alkali titanate ion exchanger has a particle size distribution d₅₀ value of between about 100 μm and about 125 μm. In an aspect, the alkali titanate ion exchanger has a particle size distribution d₅₀ value of between about 50 μm and about 90 μm. In an aspect, the alkali titanate ion exchanger has a particle size distribution d₅₀ value of between about 50 μm and about 80 μm. In an aspect, the alkali titanate ion exchanger has a particle size distribution d₅₀ value of between about 50 μm and about 70 μm. In an aspect, the alkali titanate ion exchanger has a particle size distribution d₅₀ value of between about 60 μm and about 69 μm. In an aspect, the alkali titanate ion exchanger has a particle size distribution d₅₀ value of about 66 μm. In an aspect, the alkali titanate ion exchanger has a particle size distribution d₅₀ value of between about 90 μm and about 110 μm. In an aspect, the alkali titanate ion exchanger has a particle size distribution d₅₀ value of between about 95 μm and about 105 μm. In an aspect, the alkali titanate ion exchanger has a particle size distribution d₅₀ value of about 102 μm.

In an aspect, the alkali titanate ion exchanger of the present disclosure has a particle size distribution d₉₀ value of between about 105 microns (μm) and about 190 μm. In an aspect, the alkali titanate ion exchanger has a particle size distribution d₉₀ value of between about 105 μm and about 175 μm. In an aspect, the alkali titanate ion exchanger has a particle size distribution d₉₀ value of between about 105 μm and about 160 μm. In an aspect, the alkali titanate ion exchanger has a particle size distribution d₉₀ value of between about 105 μm and about 145 μm. In an aspect, the alkali titanate ion exchanger has a particle size distribution d₉₀ value of between about 105 μm and about 130 μm. In an aspect, the alkali titanate ion exchanger has a particle size distribution d₉₀ value of between about 105 μm and about 115 μm. In an aspect, the alkali titanate ion exchanger has a particle size distribution d₉₀ value of between about 115 μm and about 190 μm. In an aspect, the alkali titanate ion exchanger has a particle size distribution d₉₀ value of between about 130 μm and about 190 μm. In an aspect, the alkali titanate ion exchanger has a particle size distribution d₉₀ value of between about 145 μm and about 190 μm. In an aspect, the alkali titanate ion exchanger has a particle size distribution d₉₀ value of between about 160 μm and about 190 μm. In an aspect, the alkali titanate ion exchanger has a particle size distribution d₉₀ value of between about 175 μm and about 190 μm. In an aspect, the alkali titanate ion exchanger has a particle size distribution d₉₀ value of between about 120 μm and about 175 μm. In an aspect, the alkali titanate ion exchanger has a particle size distribution d₉₀ value of between about 125 μm and about 135 μm. In an aspect, the alkali titanate ion exchanger has a particle size distribution d₉₀ value of about 132 μm. In an aspect, the alkali titanate ion exchanger has a particle size distribution d₉₀ value of between about 160 μm and about 170 μm. In an aspect, the alkali titanate ion exchanger has a particle size distribution d₉₀ value of about 165 μm.

In an aspect, less than 3% of the particles of the alkali titanate ion exchanger have a particle size of less than 3 μm. In an aspect, less than 2% of the particles of the alkali titanate ion exchanger have a particle size of less than 3 μm. In an aspect, less than 1% of the particles of the alkali titanate ion exchanger have a particle size of less than 3 μm. In an aspect, less than 0.5% of the particles of the alkali titanate ion exchanger have a particle size of less than 3 μm. In an aspect, less than 0.1% of the particles of the alkali titanate ion exchanger have a particle size of less than 3 μm. In an aspect, between about 3% and about 0.1% of the particles of the alkali titanate ion exchanger have a particle size of less than 3 μm. In an aspect, between about 1% and about 0.1% of the particles of the alkali titanate ion exchanger have a particle size of less than 3 μm. In an aspect, between about 0.5% and about 0.1% of the particles of the alkali titanate ion exchanger have a particle size of less than 3 μm.

In an aspect, the alkali titanate ion exchanger compositions used in the methods provided herein can be synthesized directly and/or meshed to achieve a particle size distribution (PSD) that meets the FDA standard for non-systemic solid particles (greater than 97% of the material by volume has a particle size greater than 3 micrometers). This avoids the particles perfusing out of the small intestine into the blood stream, which can result in the material accumulating in the liver and kidneys. The non-systemic alkal titanate ion exchanger compositions of the present disclosure are designed to achieve minimal side effects above that of a placebo. As described in U.S. Pat. Nos. 8,802,152, 8,808,750, 9,844,567, and 10,335,432, it has been theorized that small particles, less than 3 μm in diameter, of an insoluble powder could potentially be absorbed into a patient's bloodstream through the small intestine resulting in undesirable effects such as the accumulation of particles in the urinary tract of the patient, and particularly in the patient's kidneys. Indeed, the Label for LOKELMA® (sodium zirconium cyclosilicate), an FDA approved drug, describes it as a non-absorbed powder that includes no more than 3% of particles with a diameter below 3 μm. An in vivo pharmacokinetic study in rats showed that a dose of sodium zirconium cyclosilicate powder that meets this specification for particle size distribution was recovered in the feces with no evidence of substantial systemic absorption.

In an aspect, the alkali titanate ion exchanger of the present disclosure has a distribution coefficient (K_{d}) for Pb²⁺ of between about 100,000 milliliters per gram (mL/g) and about 2,500,000 mL/g. In an aspect, the alkali titanate ion exchanger has a K_{d} for Pb²⁺ of between about 200,000 mL/g and about 2,000,000 mL/g. In an aspect, the alkali titanate ion exchanger has a K_{d} for Pb²⁺ of between about 200,000 mL/g and about 1,600,000 mL/g. In an aspect, the alkali titanate ion exchanger has a K_{d} for Pb²⁺ of between about 200,000 mL/g and about 1,100,000 mL/g. In an aspect, the alkali titanate ion exchanger has a K_{d} for Pb²⁺ of between about 200,000 mL/g and about 1,000,000 mL/g. In an aspect, the alkali titanate ion exchanger has a K_{d} for Pb²⁺ of between about 200,000 mL/g and about 900,000 mL/g. In an aspect, the alkali titanate ion exchanger has a K_{d} for Pb²⁺ of between about 200,000 mL/g and about 800,000 mL/g. In an aspect, the alkali titanate ion exchanger has a K_{d} for Pb²⁺ of between about 200,000 mL/g and about 700,000 mL/g. In an aspect, the alkali titanate ion exchanger has a K_{d} for Pb²⁺ of between about 200,000 mL/g and about 600,000 mL/g. In an aspect, the alkali titanate ion exchanger has a K_{d} for Pb²⁺ of between about 200,000 mL/g and about 500,000 mL/g. In an aspect, the alkali titanate ion exchanger has a K_{d} for Pb²⁺ of between about 200,000 mL/g and about 400,000 mL/g. In an aspect, the alkali titanate ion exchanger has a K_{d} for Pb²⁺ of between about 400,000 mL/g and about 1,100,000 mL/g. In an aspect, the alkali titanate ion exchanger has a K_{d} for Pb²⁺ of between about 500,000 mL/g and about 1,100,000 mL/g. In an aspect, the alkali titanate ion exchanger has a K_{d} for Pb²⁺ of between about 600,000 mL/g and about 1,100,000 mL/g. In an aspect, the alkali titanate ion exchanger has a K_{d} for Pb²⁺ of between about 700,000 mL/g and about 1,100,000 mL/g. In an aspect, the alkali titanate ion exchanger has a K_{d} for Pb²⁺ of between about 800,000 mL/g and about 1,100,000 mL/g. In an aspect, the alkali titanate ion exchanger has a K_{d} for Pb²⁺ of between about 900,000 mL/g and about 1,100,000 mL/g. In an aspect, the alkali titanate ion exchanger has a K_{d} for Pb²⁺ of between about 1,000,000 mL/g and about 1,100,000 mL/g. In an aspect, the alkali titanate ion exchanger has a K_{d} for Pb²⁺ of about 856,600 mL/g. In an aspect, the alkali titanate ion exchanger has a K_{d} for Pb²⁺ of about 296,100 mL/g.

In an aspect of the methods provided herein, normal physiological levels of any one or more ions selected from Na⁺, Mg²⁺, K⁺, and Ca²⁺ are minimally disrupted in the subject in need thereof after the administration. In an aspect of the methods provided herein, the physiological levels of the one or more ions are measured in the blood of the subject in need thereof. In an aspect of the methods provided herein, normal physiological levels cations Na⁺, K⁺, Mg²⁺, and Ca²⁺ have unique concentrations in the body (blood) and unique ranges of concentrations in the body that are considered normal. Normal ranges for these cations are given in the table below. In an aspect of the methods provided herein, minimal disruption of the concentrations of these ions would be considered a change of less than about half the magnitude of the variation seen within the normal range. For instance, the normal Na⁺ concentration ranges from 310 to about 333 mg/dL, a variation of 23 mg/dL; a minimal disruption in Na⁺ concentration would in this case be less than 12 mg/dL. Minimal disruption values are given in the table below.

| | Normal Concentration | |
|---|---|---|
| Cation | Ranges in Blood | Minimal Disruption |

| | | | | |
|---|---|---|---|---|
| Na⁺ | 310-333 | mg/dL | <±12 | mg/dL |
| K⁺ | 14-20 | mg/dL | <±3.0 | mg/dL |
| Mg²⁺ | 1.5-2.6 | mg/dL | <±0.6 | mg/dL |
| Ca²⁺ | 8.5-10.5 | mg/dL | <±1.0 | mg/dL |

In an aspect, the alkali titanate ion exchanger of the present disclosure is an acid-treated alkali titanate ion exchanger. In an aspect, the alkali titanate ion exchanger of the present disclosure is an alkali titanate ion exchanger having spherical morphology or amorphous morphology. In an aspect, the alkali titanate ion exchanger of the present disclosure is an alkali titanate ion exchanger having spherical morphology. In an aspect, the alkali titanate ion exchanger of the present disclosure is an alkali titanate ion exchanger having amorphous morphology.

In an aspect, the alkali titanate ion exchanger of the present disclosure is an alkali titanate ion exchanger that is in powder form. In an aspect, the alkali titanate ion exchanger of the present disclosure is a powder. In an aspect, the alkali titanate ion exchanger of the present disclosure is in tablet form. In an aspect, the alkali titanate ion exchanger of the present disclosure is in capsule form. In an aspect, the alkali titanate ion exchanger of the present disclosure in tablet form further comprises a tablet coating.

In an aspect, the alkali titanate ion exchanger of the present disclosure is stable in a liquid environment at a pH of 1-2. In an aspect, the alkali titanate ion exchanger of the present disclosure is substantially insoluble at a pH range of 1-7. In an aspect, the alkali titanate ion exchanger of the present disclosure is substantially insoluble at a pH range of 7-13. In an aspect, the alkali titanate ion exchanger of the present disclosure is substantially insoluble at a pH range of 1-13. In an aspect, the alkali titanate ion exchanger of the present disclosure is insoluble at physiological pH. In an aspect, the alkali titanate ion exchanger of the present disclosure is substantially insoluble at physiological pH. As used herein, “physiological pH” refers to a pH range of 7.35-7.45. In an aspect, the alkali titanate ion exchanger of the present disclosure is insoluble at stomach pH. In an aspect, the alkali titanate ion exchanger of the present disclosure is substantially insoluble at stomach pH. As used herein, “stomach pH” refers to a pH range of 1-5.0. As used herein, “substantially insoluble” refers to a solubility of <1% in a fluid. According to the 2015 CRC Handbook, a material is considered soluble in a solvent if a saturated solution contains more than 1% (m/v); any material in which 1 percent or less is dissolved is considered substantially insoluble. The solubility of a compound in body fluids will be quite different than its solubility in pure water because of the effect of proteins, pH, and other solutes in body fluids. When determining solubility of a material in gastrointestinal fluids and the bloodstream the pH of the biological fluids must be considered. Gastrointestinal body fluids vary considerably in their pH (See J. Pharm. Sci., vol. 104, no. 9, pp. 2855-2863, 2015 and J. Indian Soc. Periodontol., vol. 17. No. 4, pp. 461-465, 2013). For example, the pH of the saliva is approximately neutral (mean values ranging from 6.2-7.6), stomach acid has an acidic pH (mean values ranging from 1.7-4.7), and the pH in the small intestine and colon is approximately neutral (mean values ranging from 5-8). Blood is approximately neutral, with a mean pH of about 7.4 (see Crit. Care, vol. 4, pp. 6-14, 2000). Therefore, a material is substantially insoluble in gastrointestinal bodily fluids if 1 percent or less dissolves in a simulated biological fluid across the pH range from 1.5-8. (CRC Handbook of Chemistry and Physics, 95^{th} Ed, CRC Press: Boca Raton, Fl, 2015, W. M. Haynes, Editor in Chief). In an aspect, the alkali titanate ion exchanger of the present disclosure is insoluble in one or more bodily fluids selected from the group consisting of blood, urine, and gastrointestinal fluid. In an aspect, the alkali titanate ion exchanger of the present disclosure is insoluble in blood. In an aspect, the alkali titanate ion exchanger of the present disclosure is insoluble in urine. In an aspect, the alkali titanate ion exchanger of the present disclosure is insoluble in gastrointestinal fluid. In an aspect, the alkali titanate ion exchanger of the present disclosure is insoluble in both blood and urine. In an aspect, the alkali titanate ion exchanger of the present disclosure is insoluble in both blood and gastrointestinal fluid. In an aspect, the alkali titanate ion exchanger of the present disclosure is insoluble in both urine and gastrointestinal fluid. In an aspect, the alkali titanate ion exchanger of the present disclosure is insoluble in each of blood, urine, and gastrointestinal fluid.

In an aspect, “A” of “AₘTiO_{z}” is an exchangeable cation. In an aspect, “A” is optionally selected from the group consisting of potassium ion, sodium ion, calcium ion, and hydronium ion. In an aspect, “A” is two or more selected from the group consisting of potassium ion, sodium ion, calcium ion, and hydronium ion. In an aspect, “A” is three or more selected from the group consisting of potassium ion, sodium ion, calcium ion, and hydronium ion. In an aspect, “A” is a potassium ion. In an aspect, “A” is a sodium ion. In an aspect, “A” is a calcium ion. In an aspect, “A” is a hydronium ion.

In an aspect, “m” of “AₘTiO_{z}” is the mole ratio of A to Ti. In an aspect, “m” has a value between about 0.10 to about 0.60. In an aspect, “m” has a value between about 0.10 to about 0.50. In an aspect, “m” has a value between about 0.10 to about 0.40. In an aspect, “m” has a value between about 0.10 to about 0.30. In an aspect, “m” has a value between about 0.10 to about 0.20. In an aspect, “m” has a value between about 0.20 to about 0.50. In an aspect, “m” has a value between about 0.30 to about 0.50. In an aspect, “m” has a value between about 0.40 to about 0.50.

In an aspect, Ti of “AₘTiO_{z}” comprises Ti sourced from one or more Ti-containing compound, including but not limited to TiO₂ powders such as nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof, where nano-sized is defined as a particle size of 200 nm or less, preferably 100 nm or less. In an aspect, Ti of “AₘTiO_{z}” comprises Ti in the form of preformed TiO₂ spheres sourced from one or more Ti metal sources including amorphous TiO₂, amorphous titanium oxyhydroxide, anatase titanium dioxide, rutile titanium dioxide, brookite titanium dioxide, nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof.

In an aspect, the present disclosure provides for an alkali titanate ion exchanger having an empirical formula on an anhydrous basis of: AₘTiO_{z}, wherein A is an exchangeable cation selected from sodium, lithium, potassium, magnesium, calcium, and hydronium ion and mixtures thereof; “m” is the mole ratio of A to Ti and has a value from 0.10 to 0.60; and “z” is the mole ratio of O to Ti and has a value from 2.05 to 2.60, wherein the alkali titanate ion exchanger exhibits a median particle size that is between 25 to 125 microns (μm), wherein less than 3.0% of the particles of the alkali titanate ion exchanger have a particle size less than 3 microns (μm).

In an aspect, the present disclosure provides for an alkali titanate ion exchanger having an empirical formula on an anhydrous basis of: AₘTiO_{z}, wherein A is an exchangeable cation selected from sodium, lithium, potassium, magnesium, calcium, and hydronium ion and mixtures thereof; “m” is the mole ratio of A to Ti and has a value from 0.10 to 0.60; and “z” is the mole ratio of O to Ti and has a value from 2.05 to 2.60, wherein the alkali titanate ion exchanger exhibits a median particle size that is between 25 to 125 microns (μm), wherein less than 0.5% of the particles of the alkali titanate ion exchanger have a particle size less than 3 microns (μm).

### B. Methods

In an aspect, the present disclosure provides for, and includes, a method for manufacturing a tablet or capsule for oral administration, the tablet or capsule comprising an alkali titanate ion exchanger having an empirical formula on an anhydrous basis of: AₘTiO_{z} wherein A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; “z” is the mole ratio of O to Ti and has a value from 2.05 to 2.60, wherein the alkali titanate ion exchanger exhibits a median particle size of greater than 3 microns (μm), the method comprising the steps of: (a) forming a reaction mixture comprising reactive sources of A, Ti, and water; (b) heating the reaction mixture for a period of time to form an alkali titanate ion exchanger; (c) treating the alkali titanate ion exchanger with acid or other cations by ion exchange to obtain an alkali titanate ion exchanger with the desired composition; and (d) forming a capsule or tablet comprising the alkali titanate ion exchanger, wherein the reaction mixture comprises a composition expressed in terms of mole ratios of the oxides of: p A₂O:TiO₂:f H₂O wherein “p” has a value from about 4 to 40; and “f” has a value from 20 to 1000. In an aspect, the source of Ti is TiO₂ powder or spray dried TiO₂ spheres. In an aspect, the TiO₂ powder sources include, but are not limited to nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof, where nano-sized is defined as a particle size of 200 nm or less, preferably 100 nm or less. In an aspect, Ti in the form of preformed TiO₂ spheres is sourced from amorphous TiO₂, amorphous titanium oxyhydroxide, anatase titanium dioxide, rutile titanium dioxide, brookite titanium dioxide, nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof. In an aspect, the source of Ti is TiO₂ powder. In an aspect, the source of A is sodium hydroxide, potassium hydroxide, lithium hydroxide or mixtures thereof. In an aspect, the heating is at about 85° C. to about 225° C. In an aspect, the period of time is between 0.5 days and 30 days. In an aspect, the method for manufacturing a tablet or capsule for oral administration further comprises a step of treating the alkali titanate ion exchanger with an acid or other cations by ion exchange in step (c). In an aspect, the acid is selected from the group consisting of nitric acid, hydrochloric acid, perchloric acid, and sulfuric acid. In an aspect, the acid is nitric acid. In an aspect, the acid is hydrochloric acid. In an aspect, the acid is perchloric acid. In an aspect, the acid is sulfuric acid. In an aspect, the step of treating the alkali titanate ion exchanger with an acid in (c) is at a pH of between 1 and 3 for at least 15 minutes. In an aspect, the alkali titanate may be exchanged with Mg²⁺, Ca²⁺, Na⁺, K⁺, or H⁺ or mixtures thereof, in step (c). In an aspect, the method for manufacturing a tablet or capsule for oral administration further comprises a step of sterilizing the alkali titanate ion exchanger. In an aspect, the sterilizing is by means of an autoclave. In an aspect, step (d) of forming a capsule or tablet further comprises adding a binding agent. In an aspect, the binding agent is zirconia. In an aspect, step (d) of forming a capsule or tablet further comprises annealing the alkali titanate ion exchanger. In an aspect, the method for manufacturing a tablet or capsule for oral administration further comprises a step of spray drying the alkali titanate ion exchanger to form enlarged aggregates of the alkali titanate ion exchanger of the prior to forming the capsule or tablet.

In an aspect, the present disclosure provides for, and includes, a method for selectively removing Pb²⁺ toxins from gastrointestinal fluid, the process comprising contacting the fluid containing the toxins with an alkali titanate ion exchanger, resulting in an ion exchanged ion exchanger and thereby removing the Pb²⁺ toxins from the fluid, the alkali titanate ion exchanger having an empirical formula on an anhydrous basis of: AₘTiO_{z} wherein A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60, wherein the alkali titanate ion exchanger exhibits a median particle size of greater than 3 microns (μm), and wherein the alkali titanate ion exchanger minimally disrupts the levels of any one or more ions selected from Na⁺, Mg²⁺, K⁺, and Ca²⁺ from the fluid. In an aspect, Pb²⁺ toxins are sequestered within the ion exchanged ion exchanger after the contacting. In an aspect, the method for selectively removing Pb²⁺ toxins from gastrointestinal fluid is an intracorporeal process.

In an aspect, the present disclosure provides for, and includes an intracorporeal process for removing Pb²⁺ toxins from gastrointestinal fluid, the process comprising contacting the fluid containing the toxins with an alkali titanate ion exchanger resulting in an ion exchanged ion exchanger thereby removing the toxins from the fluid, the alkali titanate ion exchanger having an empirical formula on an anhydrous basis of: AₘTiO_{z}, wherein A is an exchangeable cation selected from the group consisting of potassium ion, sodium ion, lithium ion, calcium ion, magnesium ion, hydronium ion or mixtures thereof, “m” is the mole ratio of A to Ti and has a value from 0.10 to 0.60; and “z” is the mole ratio of O to Ti and has a value from 2.05 to 2.60. In an aspect, the alkali titanate ion exchanger product has been annealed at a temperature of 350° C. for 2-6 hours. In an aspect, the alkali titanate ion exchanger has been formed into a shaped article for the purpose of oral ingestion. In an aspect, the shaped article is selected from the group consisting of pills, extrudates, spheres, pellets and irregularly shaped particles. In an aspect, the shaped article is a pill. In an aspect, the shaped article is an extrudate. In an aspect, the shaped article is a sphere. In an aspect, the shaped article is a pellet. In an aspect, the shaped article is an irregularly shaped particle.

In an aspect, the present disclosure provides for, and includes a process for preparing an alkali titanate ion exchanger, the alkali titanate ion exchanger having an empirical formula on an anhydrous basis of: AₘTiO_{z}, wherein A is an exchangeable cation selected from the group consisting of potassium ion, sodium ion, lithium ion, calcium ion, magnesium ion, hydronium ion or mixtures thereof, “m” is the mole ratio of A to Ti and has a value from 0.10 to 0.60; and “z” is the mole ratio of O to Ti and has a value from 2.05 to 2.60, the process comprising the steps of (a) forming a reaction mixture comprising reactive sources of A, Ti, and water, and (b) heating the reaction mixture at a temperature of about 85° C. to about 225° C. for a time period of 0.5 to 30 days to form the alkali titanate ion exchanger, wherein the reaction mixture comprises a composition expressed in terms of mole ratios of the oxides of:

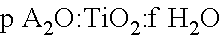

wherein “p” has a value from about 4 to 40; “a” has a value from about 0.5 to 1; and “f” has a value from 20 to 1000. In an aspect, the source of Ti is TiO₂ powder or spray dried TiO₂ spheres. In an aspect, the TiO₂ powder sources include, but are not limited to nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof, where nano-sized is defined as a particle size of 200 nm or less, preferably 100 nm or less. In an aspect, Ti in the form of preformed TiO₂ spheres is sourced from amorphous TiO₂, amorphous titanium oxyhydroxide, anatase titanium dioxide, rutile titanium dioxide, brookite titanium dioxide, nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof.

### C. Definitions

The term “and/or” when used in a list of two or more items, means that any one of the listed items can be employed by itself or in combination with any one or more of the listed items. For example, the expression “A and/or B” is intended to mean either or both of A and B, i.e., A alone, B alone, or A and B in combination. The expression “A, B and/or C” is intended to mean A alone, B alone, C alone, A and B in combination, A and C in combination, B and C in combination, or A, B, and C in combination.

As used herein, terms in the singular and the singular forms “a,” “an,” and “the,” for example, include plural referents unless the content clearly dictates otherwise.

Where a range of values is provided, it is understood that each intervening value, between the upper and lower limit of that range and any other stated or intervening value in that stated range is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure. Whenever the phrase “comprising” is used, variations such as “consisting essentially of” and “consisting of” are also contemplated.

Unless defined otherwise herein, terms are to be understood according to conventional usage by those of ordinary skill in the relevant art. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), or the “McGraw-Hill Dictionary of Scientific and Technical Terms” (6th edition, 2002, McGraw-Hill, New York).

As used herein, the term “about” refers to a range extending to +/−10% of the specified value.

As used herein, “alkali titanate” ion exchanger refers to as-synthesized alkali titanate ion exchangers as well as treated versions of these parent alkali titanates, whether the treatment is an acid treatment or an ion exchange with other allowed metals, namely lithium, sodium, potassium, magnesium, calcium, hydronium, or mixtures thereof. These materials all come under the umbrella “alkali titanate.”

As used herein, “ion exchanger” refers to a complex wherein one or more charged species may exchange with one or more charged species of the surrounding environment. In an aspect, an ion exchanger is a cation exchanger.

As used herein, “morphology” refers to form or shape of a particulate. A particulate's morphology may include, but is not limited to, spheres, interpenetrating spheres, fibers, slabs, intertwined plates, and amorphous morphology.

As used herein, “spherical morphology” refers to particulate morphology that is substantially and discernibly spherical in form.

As used herein, “amorphous morphology” refers to particulate morphology that is substantially and discernibly absent of order or repeating form.

As used herein “intracorporeal” refers to a process occurring within the body of a subject or patient. For example, an intracorporeal process for removing Pb²⁺ toxins refers to the process for removing Pb²⁺ from fluids, such as gastrointestinal fluids, within the body of a subject or patient.

As used herein “annealing” refers to a process of heat treating a material at one or more elevated temperatures for one or more pre-determined periods of time, where the annealing may alter one or more physical or chemical properties of the material, including, but not limited to, crystallinity, particle morphology, particle size distribution, and porosity. Annealing parameters/conditions may selectively alter one or more physical or chemical properties of a material and may include, but are not limited to, ramp rate, peak temperature, holding time, cooling rate, and gas environment. A gas environment for an annealing process may be selected from, but is not limited to, atmosphere, and an inert gas, such as nitrogen (N₂) or argon (Ar).

As used herein a “spray-drying” refers to a technique of forming a substantially homogenous dry powder from a liquid mixture or slurry, by drying a sprayed liquid mixture or slurry in the presence of a heated gas. The heated gas may be heated atmospheric gas or a heated inert gas, such as nitrogen (N₂) or argon (Ar).

All publications, patents, and patent applications mentioned in this disclosure are herein incorporated by reference to the same extent as if each individual publication, patent, or patent application was specifically and individually indicated to be incorporated by reference.

### EMBODIMENTS

Embodiment 1. An intracorporeal process for removing Pb²⁺ toxins from gastrointestinal fluid, the process comprising contacting the fluid containing the toxins with an alkali titanate ion exchanger resulting in an ion exchanged ion exchanger thereby removing the toxins from the fluid, the alkali titanate ion exchanger having an empirical formula on an anhydrous basis of:

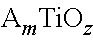

wherein
A is an exchangeable cation selected from the group consisting of potassium ion, sodium ion, lithium ion, calcium ion, magnesium ion, hydronium ion or mixtures thereof, “m” is the mole ratio of A to Ti and has a value from 0.10 to 0.60; and “z” is the mole ratio of O to Ti and has a value from 2.05 to 2.60,
   wherein the alkali titanate ion exchanger having been synthesized from a Ti source that is either nano-sized TiO₂ powder or preformed spray dried TiO₂ spheres.

Embodiment 2. The process of embodiment 1, wherein nano-sized TiO₂ powder has particle size of 200 nm or less.

Embodiment 3. The process of embodiment 1 or embodiment 2, wherein the alkali titanate ion exchanger exhibits a median particle size greater than 3 microns (μm).

Embodiment 4. The process of any one of embodiments 1 to 3, wherein the alkali titanate ion exchanger exhibits a median particle size ranging from 25 to 125 microns (μm).

Embodiment 5. The process of any one of embodiments 1 to 4, wherein less than 3% of the particles of the alkali titanate ion exchanger exhibit a particle size less than 3 microns (μm).

Embodiment 6. The process of embodiment 5, wherein less than 0.5% of the particles of the alkali titanate ion exchanger exhibit a particle size less than 3 microns (μm).

Embodiment 7. The process of any one of embodiments 1 to 6, wherein the alkali titanate ion exchanger product having been annealed at a temperature of 350° C. for 2-6 hours.

Embodiment 8. The process of any one of embodiments 1 to 7, wherein the alkali titanate ion exchanger having been formed into a shaped article to be ingested orally.

Embodiment 9. The process of embodiment 8, wherein said shaped article is selected from the group consisting of pills, extrudates, spheres, pellets and irregularly shaped particles.

Embodiment 10. A process for preparing a alkali titanate ion exchanger, the alkali titanate ion exchanger having an empirical formula on an anhydrous basis of:

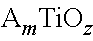

wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; “z” is the mole ratio of O to Ti and has a value from 2.05 to 2.60, the process comprising:
   (a) forming a reaction mixture comprising reactive sources of A, Ti, and water; and
   (b) heating the reaction mixture at a temperature of about 85° C. to about 225° C. for a time period of 0.5 to 30 days to form the alkali titanate ion exchanger;
      wherein the reaction mixture comprises a composition expressed in terms of mole ratios of the oxides of:

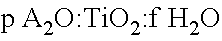

wherein “p” has a value from about 4 to 40; “a” has a value from about 0.5 to 1; and “f” has a value from 20 to 1000.

Embodiment 11. The process of embodiment 10, wherein the Ti source is selected from the group of nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof, where nano-sized is defined as a particle size of 200 nm or less, preferably 100 nm or less.

Embodiment 12. The process of embodiment 10 or embodiment 11, wherein the Ti source is preformed spray dried TiO₂ spheres.

Embodiment 13. The process of any one of embodiment 12, wherein Ti source for the spray dried TiO₂ spheres is amorphous TiO₂, amorphous titanium oxyhydroxide, anatase titanium dioxide, rutile titanium dioxide, brookite titanium dioxide, nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof.

Embodiment 14. An alkali titanate ion exchanger having an empirical formula on an anhydrous basis of:

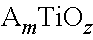

wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60,
   wherein the alkali titanate ion exchanger having been synthesized from a Ti source that is either nano-sized TiO₂ powder or preformed spray dried TiO₂ spheres, and wherein the alkali titanate ion exchanger exhibits a median particle size of greater than 3 microns (μm).

Embodiment 15. The ion exchanger of embodiment 14, wherein the alkali titanate ion exchanger is an acid-treated alkali titanate ion exchanger.

Embodiment 16. The ion exchanger of embodiment 14 or embodiment 15, wherein A is potassium ion, hydronium ion, or a mixture thereof.

Embodiment 17. The ion exchanger of any one of embodiments 14 to 16, wherein A is potassium ion.

Embodiment 18. The ion exchanger of embodiment 14 or embodiment 15, wherein A is sodium ion.

Embodiment 19. The ion exchanger of any one of embodiments 14 to 16, wherein A is a mixture of potassium and hydronium ions.

Embodiment 20. The ion exchanger of any one of embodiments 14 to 19, wherein the alkali titanate ion exchanger has spherical morphology.

Embodiment 21. The ion exchanger of any one of embodiments 14 to 19, wherein the alkali titanate ion exchanger has amorphous morphology.

Embodiment 22. The ion exchanger of any one of embodiments 14 to 21, wherein the alkali titanate ion exchanger is a powder.

Embodiment 23. The ion exchanger of any one of embodiments 14 to 22, wherein the median particle size is between 25 to 125 microns (μm).

Embodiment 24. The ion exchanger of any one of embodiments 14 to 23, wherein less than 3% of the particles of the alkali titanate ion exchanger have a particle size of less than 3 microns (μm).

Embodiment 25. The ion exchanger of embodiment 24, wherein less than 0.5% of the particles of the alkali titanate ion exchanger have a particle size less than 3 microns (μm).

Embodiment 26. The ion exchanger of any one of embodiments 14 to 25, wherein the alkali titanate ion exchanger has a particle size distribution d₁₀ value of between about 10 microns (μm) and about 80 μm.

Embodiment 27. The ion exchanger of embodiment 26, wherein the alkali ion exchanger has a particle size distribution d₁₀ value of between about 59 μm and about 65 μm.

Embodiment 28. The ion exchanger of embodiment 27, wherein the alkali titanate ion exchanger has a particle size distribution d₁₀ value of about 62 μm.

Embodiment 29. The ion exchanger of embodiment 26, wherein the alkali titanate ion exchanger has a particle size distribution d₁₀ value of between about 21 μm and about 27 μm.

Embodiment 30. The ion exchanger of embodiment 29, wherein the alkali titanate ion exchanger has a particle size distribution d₁₀ value of about 24 μm.

Embodiment 31. The ion exchanger of any one of embodiments 14 to 30, wherein the alkali titanate ion exchanger has a particle size distribution d₅₀ value of between about 45 microns (μm) and about 125 μm.

Embodiment 32. The ion exchanger of embodiment 31, wherein the alkali titanate ion exchanger has a particle size distribution d₅₀ value of between about 99 μm and about 105 μm.

Embodiment 33. The ion exchanger of embodiment 32, wherein the alkali titanate ion exchanger has a particle size distribution d₅₀ value of about 102 μm.

Embodiment 34. The ion exchanger of embodiment 31, wherein the alkali titanate ion exchanger has a particle size distribution d₅₀ value of between about 63 μm and about 69 μm.

Embodiment 35. The ion exchanger of embodiment 34, wherein the alkali titanate ion exchanger has a particle size distribution d₅₀ value of about 66 μm.

Embodiment 36. The ion exchanger of any one of embodiments 14 to 35, wherein the alkali titanate ion exchanger has a particle size distribution d₉₀ value of between about 105 microns (μm) and about 190 μm.

Embodiment 37. The ion exchanger of embodiment 36, wherein the alkali titanate ion exchanger has a particle size distribution d₉₀ value of between about 162 μm and about 168 μm.

Embodiment 38. The ion exchanger of embodiment 37, wherein the alkali titanate ion exchanger has a particle size distribution d₉₀ value of about 165 μm.

Embodiment 39. The ion exchanger of embodiment 36, wherein the alkali titanate ion exchanger has a particle size distribution d₉₀ value of between about 129 μm and about 135 μm.

Embodiment 40. The ion exchanger of embodiment 39, wherein the alkali titanate ion exchanger has a particle size distribution d₉₀ value of about 132 μm.

Embodiment 41. The ion exchanger of any one of embodiments 14 to 40, wherein the alkali titanate ion exchanger is stable in a liquid environment at a pH of 1-2.

Embodiment 42. The ion exchanger of any one of embodiments 14 to 40, wherein the alkali titanate ion exchanger is substantially insoluble at a pH range of 1-7.

Embodiment 43. The ion exchanger of any one of embodiments 14 to 40, wherein the alkali titanate ion exchanger is substantially insoluble at a pH range of 7-13.

Embodiment 44. The ion exchanger of any one of embodiments 14 to 40, wherein the alkali titanate ion exchanger is substantially insoluble at a pH range of 1-13.

Embodiment 45. The ion exchanger of any one of embodiments 14 to 40, wherein the alkali titanate ion exchanger is substantially insoluble at physiological pH.

Embodiment 46. The ion exchanger of any one of embodiments 14 to 40, and 45, wherein the alkali titanate ion exchanger is substantially insoluble at stomach pH.

Embodiment 47. The ion exchanger of any one of embodiments 14 to 46, wherein the alkali titanate ion exchanger is substantially insoluble in one or more bodily fluids.

Embodiment 48. The ion exchanger of embodiment 47, wherein the one or more bodily fluids are selected from the group consisting of blood, urine, and gastrointestinal fluid.

Embodiment 49. The ion exchanger of any one of embodiments 14 to 48, wherein the alkali titanate ion exchanger has a distribution coefficient (K_{d}) for Pb²⁺ of between about 50,000 to about 5,500,000 milliliters per gram (mL/g) in solution.

Embodiment 50. The ion exchanger of embodiment 49, wherein the alkali titanate ion exchanger has a K_{d} for Pb²⁺ of between about 100,000 to about 2,500,000 milliliters per gram (mL/g).

Embodiment 51. The ion exchanger of embodiment 50, wherein the alkali titanate ion exchanger has a K_{d} for Pb²⁺ of about 856,600.

Embodiment 52. The ion exchanger of embodiment 50, wherein the alkali titanate ion exchanger has a K_{d} for Pb²⁺ of about 296,100.

Embodiment 53. A method for manufacturing a tablet or capsule for oral administration, the tablet or capsule comprising an alkali titanate ion exchanger having an empirical formula on an anhydrous basis of:

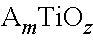

wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60, wherein the alkali titanate ion exchanger having been synthesized from a Ti source that is either nano-sized TiO₂ powder or preformed spray dried TiO₂ spheres, and wherein the alkali titanate ion exchanger exhibits a median particle size of greater than 3 microns (μm), the method comprising the steps of:
   (a) forming a reaction mixture comprising reactive sources of A, Ti, and water,
   (b) heating the reaction mixture for a period of time sufficient to form an alkali titanate ion exchanger,
   (c) optionally treating the alkali titanate ion exchanger via acid extraction and/or ion exchange with alkali metals, alkaline earth metals, or mixtures thereof to form the alkali titanate ion exchanger with the desired composition; and
   (d) forming a capsule or tablet comprising the alkali titanate ion exchanger,
      wherein the reaction mixture comprises a composition expressed in terms of mole ratios of the oxides of:

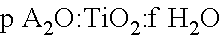

wherein “p” has a value from about 4 to 40; and “f” has a value from 20 to 1000.

Embodiment 54. The method of embodiment 53, wherein the source of Ti is TiO₂ powder, including nano-sized TiO₂, or preformed spray dried TiO₂ spheres.

Embodiment 55. The method of embodiment 54, wherein the TiO₂ powder source is selected from the group of nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof, where nano-sized is defined as a particle size of 200 nm or less, preferably 100 nm or less.

Embodiment 56. The method of embodiment 54, wherein the Ti source is preformed spray dried TiO₂ spheres.

Embodiment 57. The method of any one of embodiment 56 wherein the Ti source for preformed spray dried TiO₂ spheres is amorphous TiO₂, amorphous titanium oxyhydroxide, anatase titanium dioxide, rutile titanium dioxide, brookite titanium dioxide, nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof.

Embodiment 58. The method of any one of embodiments 53 to 57, wherein the source of A is selected from the group of sodium hydroxide, potassium hydroxide, lithium hydroxide, or mixtures thereof.

Embodiment 59. The method of any one of embodiments 53 to 58, wherein the alkali base is potassium hydroxide.

Embodiment 60. The method of any one of embodiments 53 to 59, wherein the heating is at about 85° C. to about 225° C.

Embodiment 61. The method of any one of embodiments 53 to 60, wherein the period of time is between 0.5 days and 30 days.

Embodiment 62. The method of any one of embodiments 53 to 61, further comprising a step of treating the alkali titanate ion exchanger with an acid in step (c).

Embodiment 63. The method of embodiment 62, wherein the acid is selected from the group consisting of nitric acid, hydrochloric acid, perchloric acid, and sulfuric acid.

Embodiment 64. The method of embodiment 63, wherein the acid is nitric acid.

Embodiment 65. The method of any one of embodiments 62 to 64 wherein the treating the alkali titanate ion exchanger with an acid is at a pH of between 1 and 3 for at least 15 minutes.

Embodiment 66. The method of any one of embodiments 53 to 65, further comprising a step of sterilizing the alkali titanate ion exchanger.

Embodiment 67. The method of embodiment 66, wherein the sterilizing is by means of an autoclave.

Embodiment 68. The method of any one of embodiments 53 to 67, wherein the forming the capsule or tablet further comprises adding a binding agent.

Embodiment 69. The method of embodiment 68, wherein the binding agent is zirconia.

Embodiment 70. The method of any one of embodiments 53 to 69, wherein forming the capsule or tablet comprises annealing the alkali titanate ion exchanger.

Embodiment 71. The method of any one of embodiments 53 to 70, further comprising a step of spray drying the alkali titanate ion exchanger to form enlarged aggregates of the alkali titanate ion exchanger of the prior to forming the capsule or tablet.

Embodiment 72. The method of any one of embodiments 53 to 71, wherein A is potassium ion, hydronium ion, or a mixture thereof.

Embodiment 73. The method of any one of embodiments 53 to 72, wherein A is potassium ion.

Embodiment 74. The method of any one of embodiments 53 to 72, wherein A is hydronium ion.

Embodiment 75. The method of any one of embodiments 53 to 72, wherein A is a mixture of potassium and hydronium ions.

Embodiment 76. The method of any one of embodiments 53 to 75, wherein the alkali titanate ion exchanger has spherical morphology.

Embodiment 77. The method of any one of embodiments 53 to 75, wherein the alkali titanate ion exchanger has amorphous morphology.

Embodiment 78. The method of any one of embodiments 53 to 77, wherein the alkali titanate ion exchanger is a powder.

Embodiment 79. The method of any one of embodiments 53 to 78, wherein the median particle size is between 25 to 125 microns (μm).

Embodiment 80. The method of any one of embodiments 53 to 79, wherein less than 3% of the particles of the alkali titanate ion exchanger have a particle size of less than 3 microns (μm).

Embodiment 81. The method of embodiment 80, wherein less than 0.5% of the particles of the alkali titanate ion exchanger have a particle size less than 3 microns (μm).

Embodiment 82. The method of any one of embodiments 53 to 81, wherein the alkali titanate ion exchanger has a particle size distribution d₁₀ value of between about 10 microns (μm) and about 80 μm.

Embodiment 83. The method of embodiment 82, wherein the alkali titanate ion exchanger has a particle size distribution d₁₀ value of between about 59 μm and about 65 μm.

Embodiment 84. The method of embodiment 83, wherein the alkali titanate ion exchanger has a particle size distribution d₁₀ value of about 62 μm.

Embodiment 85. The method of embodiment 82, wherein the alkali titanate ion exchanger has a particle size distribution d₁₀ value of between about 21 μm and about 27 μm.

Embodiment 86. The method of embodiment 85, wherein the alkali titanate ion exchanger has a particle size distribution d₁₀ value of about 24 μm.

Embodiment 87. The method of any one of embodiments 53 to 86, wherein the alkali titanate ion exchanger has a particle size distribution d₅₀ value of between about 45 microns (μm) and about 125 μm.

Embodiment 88. The method of embodiment 87, wherein the alkali titanate ion exchanger has a particle size distribution d₅₀ value of between about 99 μm and about 105 μm.

Embodiment 89. The method of embodiment 88, wherein the alkali titanate ion exchanger has a particle size distribution d₅₀ value of about 102 μm.

Embodiment 90. The method of embodiment 87, wherein the alkali titanate ion exchanger has a particle size distribution d₅₀ value of between about 63 μm and about 69 μm.

Embodiment 91. The method of embodiment 90, wherein the alkali titanate ion exchanger has a particle size distribution d₅₀ value of about 66 μm.

Embodiment 92. The method of any one of embodiments 53 to 91, wherein the alkali titanate ion exchanger has a particle size distribution d₉₀ value of between about 105 microns (μm) and about 190 μm.

Embodiment 93. The method of embodiment 92, wherein the alkali titanate ion exchanger has a particle size distribution d₉₀ value of between about 162 μm and about 168 μm.

Embodiment 94. The method of embodiment 93, wherein the alkali titanate ion exchanger has a particle size distribution d₉₀ value of about 165 μm.

Embodiment 95. The method of embodiment 92, wherein the alkali titanate ion exchanger has a particle size distribution d₉₀ value of between about 129 μm and about 135 μm.

Embodiment 96. The method of embodiment 95, wherein the alkali titanate ion exchanger has a particle size distribution d₉₀ value of about 132 μm.

Embodiment 97. The method of any one of embodiments 53 to 96, wherein the alkali titanate ion exchanger is stable in a liquid environment at a pH of 1-2.

Embodiment 98. The method of any one of embodiments 53 to 97, wherein the alkali titanate ion exchanger is substantially insoluble at a pH range of 1-7.

Embodiment 99. The method of any one of embodiments 53 to 97, wherein the alkali titanate ion exchanger is substantially insoluble at a pH range of 7-13.

Embodiment 100. The method of any one of embodiments 53 to 97, wherein the alkali titanate ion exchanger is substantially insoluble at a pH range of 1-13.

Embodiment 101. The method of any one of embodiments 53 to 97, wherein the alkali titanate ion exchanger is substantially insoluble at physiological pH.

Embodiment 102. The method of any one of embodiments 53 to 97, and 101, wherein the alkali titanate ion exchanger is substantially insoluble at stomach pH.

Embodiment 103. The method of any one of embodiments 53 to 102, wherein the alkali titanate ion exchanger is substantially insoluble in one or more bodily fluids.

Embodiment 104. The method of embodiment 103, wherein the one or more bodily fluids are selected from the group consisting of blood, urine, and gastrointestinal fluid.

Embodiment 105. The method of any one of embodiments 53 to 104, wherein the alkali titanate ion exchanger has a distribution coefficient (K_{d}) for Pb²⁺ of between about 50,000 to about 5,500,000 milliliters per gram (mL/g) in solution.

Embodiment 106. The method of embodiment 105, wherein the alkali titanate ion exchanger has a K_{d} for Pb²⁺ of between about 100,000 to about 2,500,000 milliliters per gram (mL/g).

Embodiment 107. The method of embodiment 105, wherein the alkali titanate ion exchanger has a K_{d} for Pb²⁺ of about 856,600.

Embodiment 108. The method of embodiment 105, wherein the alkali titanate ion exchanger has a K_{d} for Pb²⁺ of about 296,100.

Embodiment 109. A method for selectively removing Pb²⁺ toxins from gastrointestinal fluid, the process comprising contacting the fluid containing the toxins with an alkali titanate ion exchanger, resulting in an ion exchanged ion exchanger and thereby removing the Pb²⁺ toxins from the fluid, the alkali titanate ion exchanger having an empirical formula on an anhydrous basis of:

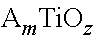

wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60,
   wherein the alkali titanate ion exchanger having been synthesized from a Ti source that is either nano-sized TiO₂ powder or preformed spray dried TiO₂ spheres, and wherein the alkali titanate ion exchanger exhibits a median particle size of greater than 3 microns (μm), and wherein the alkali titanate ion exchanger minimally disrupts the levels of any one or more ions selected from Na⁺, Mg²⁺, K⁺, and Ca²⁺ from the fluid.

Embodiment 110. The method of embodiment 109, wherein the alkali titanate ion exchanger is an acid-treated alkali titanate ion exchanger.

Embodiment 111. The method of embodiment 109 or embodiment 110, wherein A is potassium ion, hydronium ion, or a mixture thereof.

Embodiment 112. The method of any one of embodiments 109 to 111, wherein A is potassium ion.

Embodiment 113. The method of any one of embodiments 109 to 111, wherein A is hydronium ion.

Embodiment 114. The method of any one of embodiments 109 to 111, wherein A is a mixture of potassium and hydronium ions.

Embodiment 115. The method of any one of embodiments 109 to 114, wherein the alkali titanate ion exchanger has a spherical morphology.

Embodiment 116. The method of any one of embodiments 109 to 114, wherein the alkali titanate ion exchanger has an amorphous morphology.

Embodiment 117. The method of any one of embodiments 109 to 116, wherein the alkali titanate ion exchanger is a powder.

Embodiment 118. The method of any one of embodiments 109 to 117, wherein the median particle size is between 25 to 125 microns (μm).

Embodiment 119. The method of any one of embodiments 109 to 118, wherein less than 3% of the particles of the alkali titanate ion exchanger exhibit a particle size less than 3 microns (μm).

Embodiment 120. The method of embodiment 119, wherein less than 0.5% of the particles of the alkali titanate ion exchanger exhibit a particle size less than 3 microns (μm).

Embodiment 121. The method of any one of embodiments 109 to 120, wherein the alkali titanate ion exchanger has a particle size distribution d₁₀ value of between about 10 microns (μm) and about 80 μm.

Embodiment 122. The method of embodiment 121, wherein the alkali titanate ion exchanger has a particle size distribution d₁₀ value of between about 59 μm and about 65 μm.

Embodiment 123. The method of embodiment 122, wherein the alkali titanate ion exchanger has a particle size distribution d₁₀ value of about 62 μm.

Embodiment 124. The method of embodiment 121, wherein the alkali titanate ion exchanger has a particle size distribution d₁₀ value of between about 21 μm and about 27 μm.

Embodiment 125. The method of embodiment 124, wherein the alkali titanate ion exchanger has a particle size distribution d₁₀ value of about 24 μm.

Embodiment 126. The method of any one of embodiments 109 to 125, wherein the alkali titanate ion exchanger has a particle size distribution d₅₀ value of between about 45 microns (μm) and about 125 μm.

Embodiment 127. The method of embodiment 126, wherein the alkali titanate ion exchanger has a particle size distribution d₅₀ value of between about 99 μm and about 105 μm.

Embodiment 128. The method of embodiment 127, wherein the alkali titanate ion exchanger has a particle size distribution d₅₀ value of about 102 μm.

Embodiment 129. The method of embodiment 126, wherein the alkali titanate ion exchanger has a particle size distribution d₅₀ value of between about 63 μm and about 69 μm.

Embodiment 130. The method of embodiment 129, wherein the alkali titanate ion exchanger has a particle size distribution d₅₀ value of about 66 μm.

Embodiment 131. The method of any one of embodiments 109 to 130, wherein the alkali titanate ion exchanger has a particle size distribution d₉₀ value of between about 105 microns (μm) and about 190 μm.

Embodiment 132. The method of embodiment 131, wherein the alkali titanate ion exchanger has a particle size distribution d₉₀ value of between about 162 μm and about 168 μm.

Embodiment 133. The method of embodiment 132, wherein the alkali titanate ion exchanger has a particle size distribution d₉₀ value of about 165 μm.

Embodiment 134. The method of embodiment 131, wherein the alkali titanate ion exchanger has a particle size distribution d₉₀ value of between about 129 μm and about 135 μm.

Embodiment 135. The method of embodiment 134, wherein the alkali titanate ion exchanger has a particle size distribution d₉₀ value of about 132 μm.

Embodiment 136. The method of any one of embodiments 109 to 135, wherein the alkali titanate ion exchanger is stable in a liquid environment at a pH of 1-2.

Embodiment 137. The method of any one of embodiments 109 to 136, wherein the alkali titanate ion exchanger is substantially insoluble at a pH range of 1-7.

Embodiment 138. The method of any one of embodiments 109 to 136, wherein the alkali titanate ion exchanger is substantially insoluble at a pH range of 7-13.

Embodiment 139. The method of any one of embodiments 109 to 136, wherein the alkali titanate ion exchanger is substantially insoluble at a pH range of 1-13.

Embodiment 140. The method of any one of embodiments 109 to 139, wherein the alkali titanate ion exchanger is substantially insoluble in the fluid.

Embodiment 141. The method of any one of embodiments 109 to 140, wherein the alkali titanate ion exchanger has a distribution coefficient (K_{d}) for Pb²⁺ of between about 50,000 to about 5,500,000 milliliters per gram (mL/g).

Embodiment 142. The method of embodiment 141, wherein the alkali titanate ion exchanger has a K_{d} for Pb²⁺ of between about 100,000 to about 2,500,000 mL/g.

Embodiment 143. The method of embodiment 142, wherein the alkali titanate ion exchanger has a K_{d} for Pb²⁺ of about 856,600.

Embodiment 144. The method of embodiment 142, wherein the alkali titanate ion exchanger has a K_{d} for Pb²⁺ of about 296,100.

Embodiment 145. The method of any one of embodiments 109 to 144, wherein the alkali titanate ion exchanger minimally disrupts the levels of any one or more ions selected from Na⁺, Mg²⁺, K⁺, and Ca²⁺.

Embodiment 146. The method of embodiment 145, wherein the alkali titanate ion exchanger reduces the levels of Na⁺ ions by 12 mg/dL or less.

Embodiment 147. The method of embodiment 145, wherein the alkali titanate ion exchanger reduces the levels of K⁺ ions by 3.0 mg/dL or less.

Embodiment 148. The method of embodiment 145, wherein the alkali titanate ion exchanger reduces the levels of Mg²⁺ ions by 0.6 mg/dL or less.

Embodiment 147. The method of embodiment 145, wherein the alkali titanate ion exchanger reduces the levels of Ca²⁺ ions by 1.0 mg/dL or less.

Embodiment 148. The method of embodiment 109 to 145, wherein the alkali titanate ion exchanger does not substantially reduce the levels of any one or more ions selected from Na⁺, Mg²⁺, K⁺, and Ca²⁺.

Embodiment 149. The method of any one of embodiments 109 to 148, wherein the one or more ions are Na⁺, Mg²⁺, K⁺, and Ca²⁺.

Embodiment 150. The method of any one of embodiments 109 to 149, wherein the Pb²⁺ toxins are sequestered within the ion exchanged ion exchanger after the contacting.

Embodiment 151. The method of any one of embodiments 109 to 150, wherein the selectively removing is an intracorporeal process.

### EXAMPLES

The x-ray patterns presented in the following examples were obtained using standard x-ray powder diffraction techniques. The radiation source was a high-intensity, x-ray tube operated at 45 kV and 35 mA. The diffraction pattern from the copper K-alpha radiation was obtained by appropriate computer-based techniques. Flat compressed powder samples were continuously scanned at 2° to at least 56° (2θ). Interplanar spacings (d) in Angstrom units were obtained from the position of the diffraction peaks expressed as θ where θ is the Bragg angle as observed from digitized data. Intensities were determined from the integrated area of diffraction peaks after subtracting background, “I” being the intensity of the strongest line or peak, and “I” being the intensity of each of the other peaks.

As will be understood by those skilled in the art the determination of the parameter 20 is subject to both human and mechanical error, which in combination can impose an uncertainty of about ±0.4° on each reported value of 2θ. This uncertainty is, of course, also manifested in the reported values of the d-spacings, which are calculated from the 2θ values. This imprecision is general throughout the art and is not sufficient to preclude the differentiation of the present crystalline materials from each other and from the compositions of the prior art. In the x-ray patterns reported, the relative intensities of the d-spacings are indicated by the notations vs, s, m, and w which represent very strong, strong, medium, and weak, respectively. In terms of 100×I/Iₒ, the above designations are defined as:

$$w > 0 - 15 ; m > 15 - 60 : s > 60 - 80 and vs > 80 - 100$$

In certain instances, the purity of a synthesized product may be assessed with reference to its x-ray powder diffraction pattern. Thus, for example, if a sample is stated to be pure, it is intended only that the x-ray pattern of the sample is free of lines attributable to crystalline impurities, not that there are no amorphous materials present.

Additionally, all elemental analyses will report the metals stoichiometry, the elements oxygen and hydrogen and water were not determined by analysis. Oxygen stoichiometry is inferred by balancing the charges on the metals, the compositions are described in their anhydrous state.

To illustrate the instant invention more fully, the following examples are set forth. It is to be understood that the examples are only by way of illustration and are not intended as an undue limitation on the broad scope of the invention as set forth in the appended claims.

### Comparative Examples C1-C5

The following Comparative Examples are put forth to better illustrate the advances of the current invention over inventions in the prior art and other alternatives.

### Example C1

A sample of sodium nonatitanate from Allied-Signal was disclosed for Pb²⁺ uptake from bodily fluids in U.S. Pat. No. 11,964,266, Example 4. Characterization of the sample by PXRD was consistent with sodium nonatitanate. Representative x-ray diffraction lines for the sample are given in Table 1 below. Elemental analysis by ICP yielded the metals stoichiometry Na_{0.5}Ti.

TABLE 1

| 2-Θ | d(Å) | I/I₀ % |
|---|---|---|
| 9.22 | 9.58 | vs |
| 10.04 | 8.80 | w |
| 24.03 | 3.701 | w |
| 28.07 | 3.18 | w (br) |
| 33.66 | 2.66 | w (br) |
| 39.57 | 2.28 | w (br) |
| 48.08 | 1.89 | w (br) |

### Example C2

A commercial potassium octatitanate, product number HON393 from Honeywell Specialty Chemicals, Seelze GMBH was disclosed for Pb uptake from bodily fluids in U.S. Pat. No. 11,964,266, Example 17. This sample is a composite consisting of potassium octatitanate, K₂Ti₈O₁₇, but also containing anatase, and potassium hexatitanate, K₂Ti₆O₁₃. The sample was characterized by PXRD for which representative x-ray diffraction lines are shown in Table 2. Elemental analyses by ICP yielded metals stoichiometry K_{0.26}Ti, consistent with the expected stoichiometry of potassium octatitanate, K₂Ti₈O₁₇.

TABLE 2

| 2-Θ | d(Å) | I/I₀ % |
|---|---|---|
| 11.34 | 7.80 | vs |
| 24.00 | 3.70 | w |
| 25.32 | 3.51 | vs* |
| 29.09 | 3.07 | s |
| 29.68 | 3.01 | w (sh) |
| 36.96 | 2.43 | w* |
| 37.81 | 2.38 | m* |
| 38.60 | 2.33 | w* |
| 47.76 | 1.90 | m |
| 48.04 | 1.89 | m* |
| 53.90 | 1.70 | m* |
| 55.08 | 1.67 | m |
| 62.18 | 1.49 | w* |
| 62.68 | 1.48 | m* |
| 68.76 | 1.36 | w |

*anatase contributions

### Example C3

A 50 ml polypropylene beaker was charged with 10 g of pre-made 8.5 M KOH solution and stirred with a stirbar. With vigorous mixing, 1 g of mixed rutile/anatase TiO₂ powder was added. After homogenization, the reaction mixture was placed in a 45 ml Teflon-lined Parr vessel and digested quiescently at 175° C. for 24 hrs at autogenous pressure. The solid product was isolated by centrifugation, washed with deionized water, and dried at 100° C. overnight. PXRD was used to characterize the product, representative diffraction lines are listed in Table 3 below.

TABLE 3

| 2-Θ | d(Å) | I/I₀ % |
|---|---|---|
| 10.96 | 8.07 | m |
| 24.20 | 3.67 | m |
| 29.23 | 3.05 | vs |
| 33.46 | 2.68 | w |
| 42.72 | 2.11 | m |
| 47.98 | 1.89 | s |
| 59.34 | 1.56 | w |
| 66.01 | 1.41 | w |

### Example C4

A 100 ml polypropylene beaker was charged with 46 g of pre-made 7.5 M NaOH solution and placed under an overhead mixer. With vigorous mixing, 4 g of pure anatase TiO₂ powder (Sigma-Aldrich) was added, allowing the reaction mixture to stir for 5 minutes. A portion of the reaction mixture was placed in a 45 ml Teflon-lined Parr vessel and digested quiescently at 200° C. for 24 hrs at autogenous pressure. The solid product was isolated by centrifugation, washed with deionized water, and dried at 100° C. overnight. PXRD was used to characterize the product, representative diffraction lines are listed in Table 4 below.

TABLE 4

| 2-Θ | d(Å) | I/I₀ % | |
|---|---|---|---|
| 9.18 | 9.62 | vs | |
| 10.24 | 8.63 | w | (sh) |
| 18.34 | 4.83 | w | |
| 24.24 | 3.67 | w | |
| 28.29 | 3.15 | w | (br) |

| | | |
|---|---|---|
| 47.62 | 1.91 | w |
| 48.10 | 1.89 | m |

### Example C5

A 100 ml polypropylene beaker was charged with 40 g of pre-made 8.5 M KOH solution and placed under an overhead mixer. With vigorous mixing, 4 g of pure anatase TiO₂ powder (Sigma-Aldrich) was added, allowing the reaction mixture to stir for 5 minutes. A portion of the reaction mixture was placed in a 45 ml Teflon-lined Parr vessel and digested quiescently at 200° C. for 24 hrs at autogenous pressure. The solid product was isolated by centrifugation, washed with deionized water, and dried at 100° C. overnight. PXRD was used to characterize the product, representative diffraction lines are listed in Table 5 below.

TABLE 5

| 2-Θ | d(Å) | I/I₀ % | |
|---|---|---|---|
| 7.30 | 12.11 | w | (br) |
| 11.01 | 8.03 | w | (br) |
| 24.07 | 3.70 | w | |
| 25.30 | 3.52 | w | |
| 29.19 | 3.06 | vs | (br) |
| 31.43 | 2.84 | w | (sh) |
| 33.78 | 2.65 | m | (br) |
| 42.62 | 2.12 | w | (br) |

| | | |
|---|---|---|
| 47.70 | 1.90 | s |

### Example 1

A 100 ml polypropylene beaker was charged with 40 g of pre-made 8.5 M KOH solution and placed under an overhead mixer. With vigorous mixing, 4 g of pure anatase nano-sized TiO₂ powder (Kemira), was added, allowing the reaction mixture to stir for 5 minutes. A portion of the reaction mixture was placed in a 45 ml Teflon-lined Parr vessel and digested quiescently at 200° C. for 24 hrs at autogenous pressure. The solid product was isolated by centrifugation, washed with deionized water, and dried at 100° C. overnight. PXRD was used to characterize the product, representative diffraction lines are listed in Table 6 below.

TABLE 6

| 2-Θ | d(Å) | I/I₀ % |
|---|---|---|
| 11.04 | 8.01 | w |
| 24.11 | 3.69 | m |
| 29.13 | 3.06 | vs |
| 29.41 | 3.03 | vs |
| 33.70 | 2.66 | m |
| 42.65 | 2.12 | m |
| 47.82 | 1.90 | s |
| 59.31 | 1.56 | w |
| 65.98 | 1.41 | w |

### Example 2

### Synthesis of Spray Dried TiO₂ Spheres

Spray dried TiO₂ spheres are synthesized with the Yamato Spray Drier Model DL-41. Typically, 500 grams of a slurry with 20 wt. % titanium dioxide powder in DI H₂O is prepared. To 400 g of deionized water, 100 g of fumed TiO₂ powder (mixed rutile/anatase phase) is added while mixing with an overhead mixer at 500 RPM. The mixture is stirred for 10 minutes. The suspension is then Eiger milled for 15 minutes before spray drying. Larger agglomerates are removed by passing the suspension through a 100-mesh (150 μm) sieve. The suspension is stirred continuously to avoid settling of particles. The spray drier chamber temperature reached 110° C. with a drying air flow of 80 SCFH. The aspirator flow is 0.8 cm³/min with 10 psi head pressure. The slurry feeding speed used for this process is 16 cc/min. The collected product is screened through 60-mesh (250 μm), 100-mesh (150 μm), and 200-mesh (75 μm) sieves; the final sample collected has particles finer than 200-mesh (75 μm). Spray dried TiO₂ spheres from a typical preparation were characterized by PXRD, revealing a mixture of anatase and rutile TiO₂ topologies that were in the starting material. Characteristic diffraction lines are shown in Table 7 below, revealing the rutile and anatase components.

TABLE 7

| 2-Θ | d(Å) | I/I₀ % | |
|---|---|---|---|
| 25.24 | 3.53 | vs | (A) |
| 27.40 | 3.25 | m | (R) |
| 36.04 | 2.49 | w | (R) |
| 36.93 | 2.43 | w | (A) |
| 37.73 | 2.38 | m | (A) |
| 38.56 | 2.33 | w | (A) |
| 39.19 | 2.30 | w | (R) |
| 41.18 | 2.19 | w | (R) |
| 43.95 | 2.06 | w | (R) |
| 48.00 | 1.89 | m | (A) |
| 53.89 | 1.70 | m | (A) |
| 54.28 | 1.69 | m | (R) |
| 55.02 | 1.67 | m | (A) |
| 56.60 | 1.62 | w | (R) |
| 62.09 | 1.49 | w | (A) |
| 62.66 | 1.48 | m | (A, R) |
| 64.04 | 1.45 | w | (A) |
| 68.75 | 1.36 | w | (R) |
| 68.93 | 1.36 | w | (A) |
| 69.82 | 1.35 | w | (A) |

A = Anatase, R = Rutile

These spray dried TiO₂ spheres were used as the titania source in the following reaction. A 100 ml polypropylene beaker was charged with 40 g of 8.5 M KOH solution and placed under an overhead mixer. With vigorous mixing, 4 g of spray dried TiO₂ spheres was added and the reaction mixture allowed to stir for 5 minutes. A portion of the reaction mixture was placed in a 45 ml Teflon-lined Parr vessel and digested at 225° C. for 1-day during which it was tumbled at 30 rpm at autogenous pressure. The solid product was isolated by centrifugation, washed with deionized water, and dried at 100° C. overnight. PXRD was used to characterize the products. Representative diffraction lines for the product are listed in Table 8 below.

TABLE 8

| 2-Θ | d(Å) | I/I₀ % | |
|---|---|---|---|
| 10.96 | 8.07 | vs | (br) |
| 24.13 | 3.69 | w | |
| 29.25 | 3.05 | vs | (br) |
| 32.32 | 2.77 | m | (sh) |
| 33.56 | 2.67 | m | (br) |
| 42.48 | 2.13 | w | (br) |
| 47.92 | 1.90 | m | |
| 52.91 | 1.73 | w | (br) |
| 59.29 | 1.56 | w | (br) |
| 66.15 | 1.41 | w | (br) |

### Example 3 SEMs of the Titanate-Based Ion Exchangers

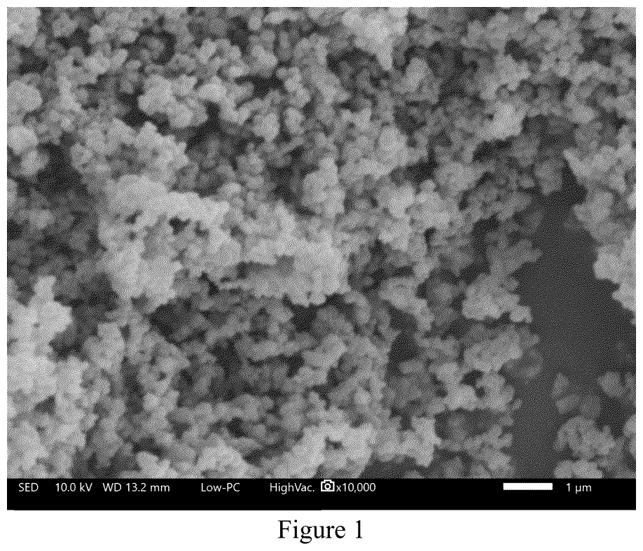

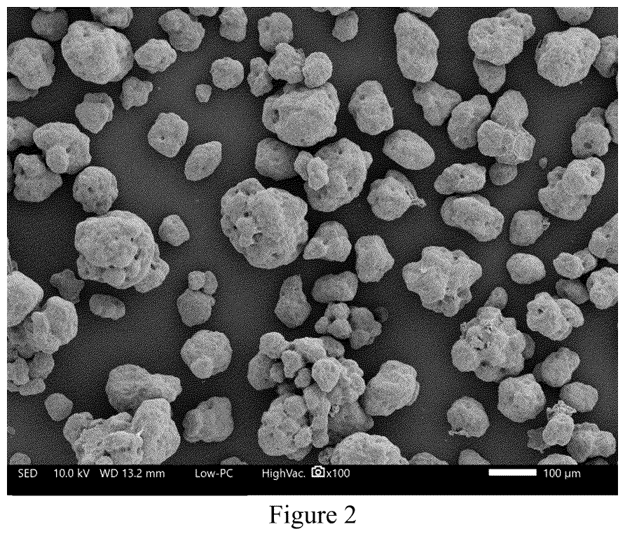

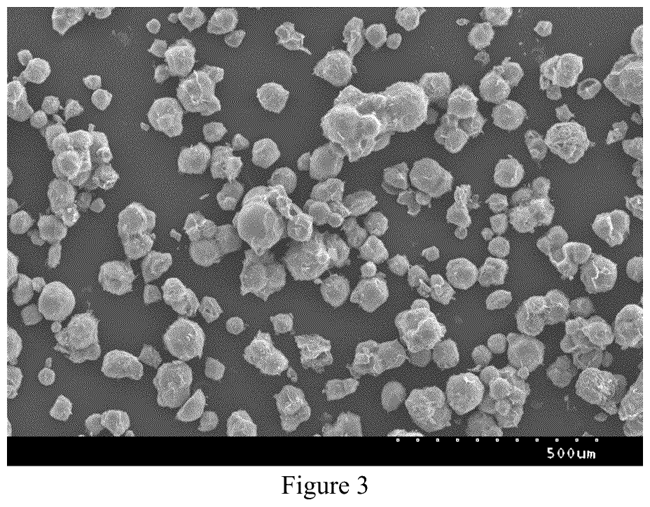

Scanning Electron Microscopy (SEM) was used to illustrate the properties of the nanotitania reagent and the resulting alkali titanate. FIG. 1 shows an SEM of the nanotitania reagent. It is seen that the sample is comprised rather uniform crystallites less than 200 nm in diameter, most less than about 100 nm across. When this titania source is digested in KOH solution at 200° C. for one day, the potassium titanates seen in FIG. 2 are formed. These relatively amorphous boulders vary in diameter from about 30 to 200 μm, large enough to avoid absorption in the gastrointestinal tract. The SEM in FIG. 3 is that of preformed spray dried titania spheres digested in 8.5 M KOH. Most of the resulting alkali titanate ion exchanger spheres are visibly between about 30 μm and 100 μm in diameter, large enough to avoid absorption in the gastrointestinal tract.

### Example 4 Particle Size Distribution

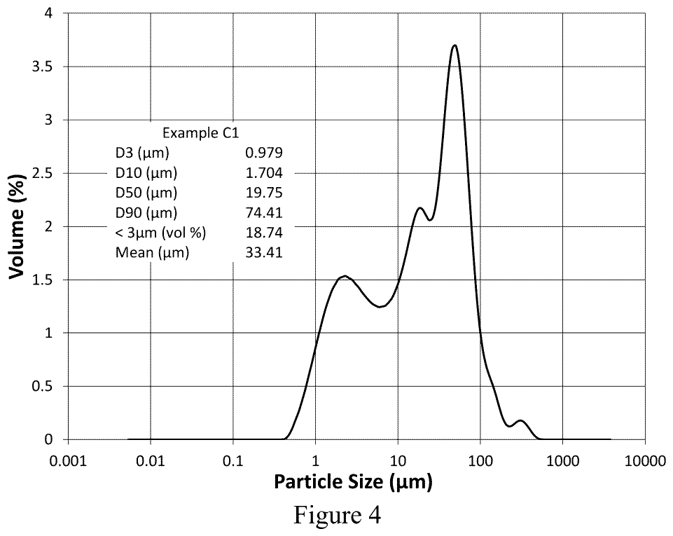

Particle size distribution (PSD) measurements were also carried out to determine the size characteristics of selected examples as well as some of the comparative examples. Separate batches of powder samples were analyzed using light scatter diffraction techniques in a LS 13 320 XR Particle Size Analyzer. Powder samples were dispersed in water and sonicated briefly before measurement. The particle size distribution and other measured parameters are shown in FIGS. 4-10. The D(3), D(10), D(50), and D(90) values represent the particle size values in microns under which smallest 3%, 10%, 50%, and 90% of the sample occur in the particle size distribution. The D(50) value is equivalent to the Median of the particle size distribution. It has been proposed that particles less than 3 microns in size can be absorbed by the body and it is desirable to have few of these particles, although 3 percent has been seen as acceptable upper limit. In the figures, the parameter<3 μm (vol %) gives the volume percent of the sample having a particle size less than 3 μm. The Mean or average particle size is also reported for each sample.

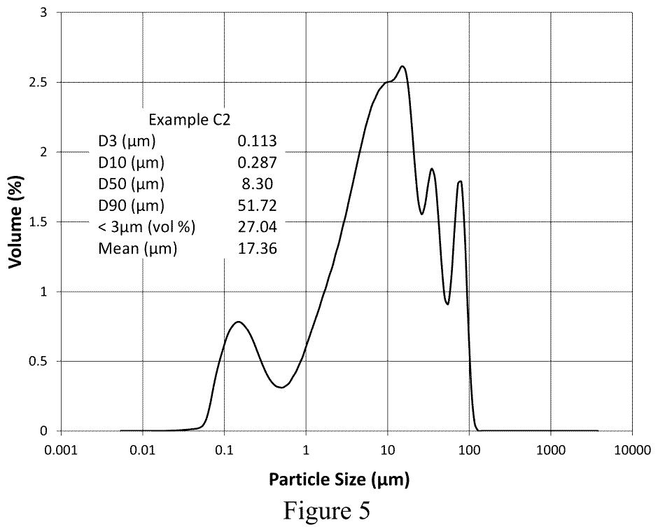

FIG. 4 shows the PSD of comparative Example C1, an Allied-Signal sodium nonatitanate sample disclosed in U.S. Pat. No. 11,964,266, Example 4, for removal of Pb and other metals from bodily fluids. For this sample, D(3) shows that 3 volume percent of the sample has a particle size less than 0.98 μm and that 18.74 volume percent of the sample has a particle size less than 3 microns. Similarly, FIG. 5 shows the particle size distribution of comparative Example C2, a Honeywell potassium octatitanate product disclosed in U.S. Pat. No. 11,964,266, Example 17, for removal of Pb and other metals from bodily fluids. For this sample, D(3) shows that 3 volume percent of the sample has a particle size less than 0.11 micron and 27.04 volume percent of the sample is less than 3 microns in particle size. For each of the comparative Example C1 and Example C2 materials, the particle size distribution is multipeaked and broad, having relatively significant fractions of sample at particle sizes ranging over two orders of magnitude. The broad distributions are characterized by mean particle sizes that are nearly twice that of the median particle sizes. In addition, for each of these samples, the large fractions of sample with particle size less than 3 μm, 18.74 and 27.04 volume percent for the Example C1 and C2 samples, respectively, makes these samples unacceptable for treatment involving the gastrointestinal tract, because these small particles could be absorbed by the body, causing adverse effects.

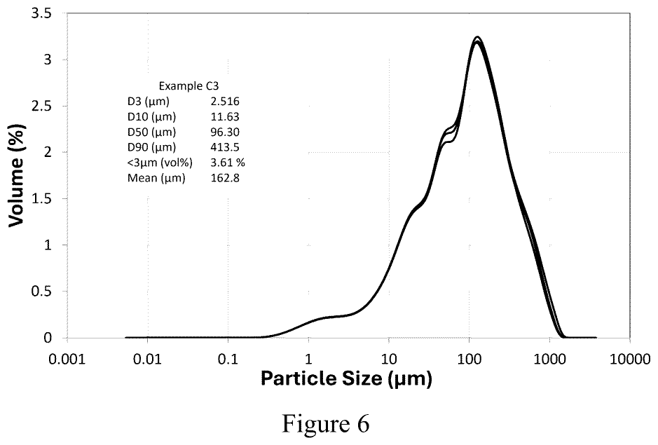

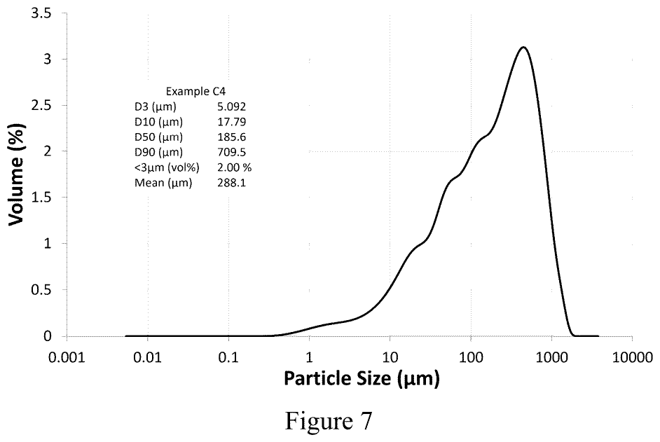

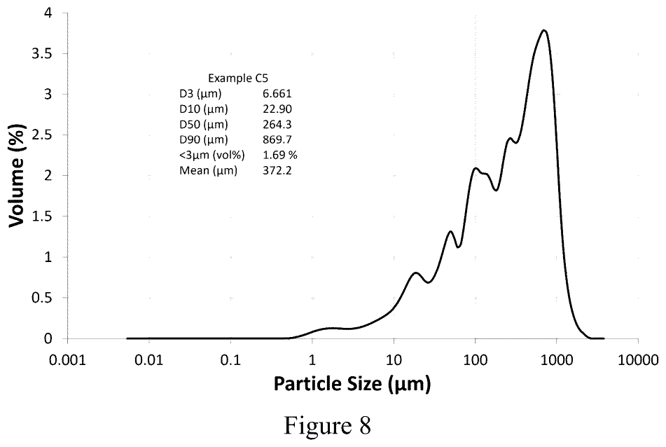

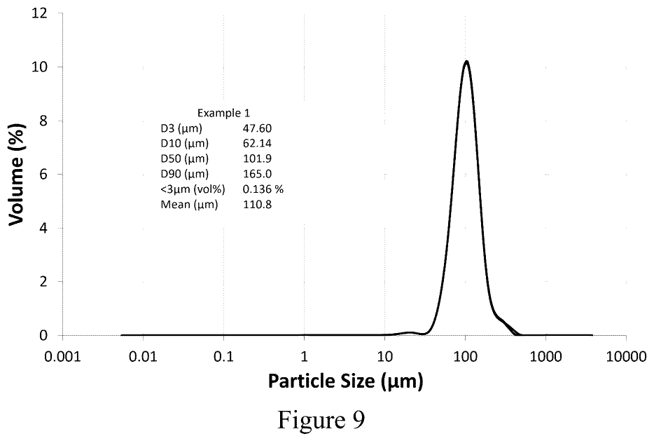

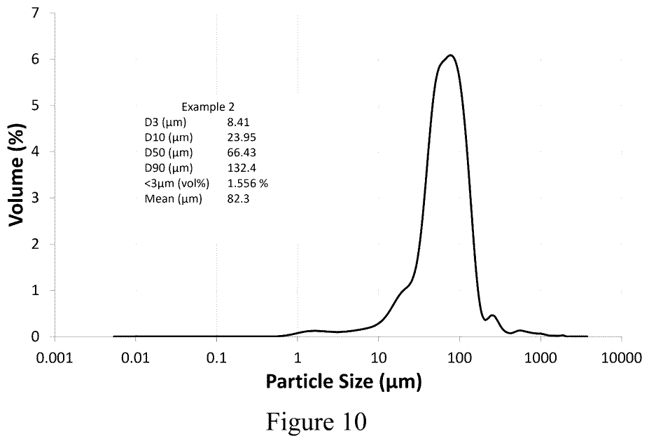

The particle size distribution of the Example C3 material is shown in FIG. 6. This sample was prepared from a mixed phase rutile/anatase titania source. The PSD seen in FIG. 6 is not as erratic at those for the Example C1 and C2 samples, resembling a broad peaked mountain. The particles in this sample are much larger getting to over a millimeter in diameter and median particle sizes are 5-10 times that seen for the Example C1 and C2 samples. While only 2.52 volume percent of the sample had diameter less than 3 μm, meeting a size standard regarding absorption in the gastrointestinal tract, the uniformity of the sample is poor, especially regarding large particle sizes. The Example C4 and C5 materials are prepared from pure anatase titania, hydrothermally converted in NaOH and KOH solutions, respectively. The PSDs for Examples C4 (FIG. 7) and C5 (FIG. 8) resemble that of Example C3, very broad and non-uniform. Particulates occur over a millimeter in size again and the profile resembles a broad mountain skewed toward the large particle size, multi-stepped in the case of Example C4 and multi-peaked in the case of example C5. These two materials also meet the size standard (less than 3 volume percent less than 3 μm) regarding absorption in the gastrointestinal tract, as D(3) for Examples C4 and C5 are 5.092 and 6.661 μm, respectively. However, the non-uniformity, especially the large size variations, can affect performance of the product. In contrast, FIG. 9 shows the PSD of the Example 1 material. This material was prepared from the pure anatase nanotitania reagent shown in FIG. 1. The PSD is visibly superior for this material, a narrow single-peaked distribution is observed, with the mean, 110.8 μm very close to the median particle size, 101.9 μm. There are very few large or small particles, D(3)=47.60 μm, while D(90)=165 μm. The D(90) is 3-5 times smaller than that of the D(90)s seen for the materials of Examples C3, C4 and C5. This material easily meets the size standard for use in the gastrointestinal tract as D(3)=47.60 μm is 15 times that of the 3 μm requirement. The SEM of the Example 1 material is seen in FIG. 2. In FIG. 10, the PSD of an alkali titanate synthesized from preformed spray dried spheres is shown. This sample is also characterized by a narrower PSD as D(3)=8.41 μm, D(50)=66.43 μm, and D(90)=132.4 μm, with fewer very small and very large particles than seen in the alkali titanates of the comparative examples.

### Example 5 Pb²⁺ Uptake

The titanate-based materials disclosed in Examples C1, C2 and Example 1 were tested to determine their ability to adsorb Pb²⁺, Mg²⁺, Ca²⁺, K⁺ and Na⁺ ions from a test solution by determining the distributions (K_{d}) for each of the metals between adsorption on the solid vs. remaining in the solution state. The test solution was prepared from the source compounds given in Table 9:

TABLE 9

| | [Mⁿ⁺] |
|---|---|
| compound | ppm |
| Ca(NO₃)₂•4H₂O | 25 |
| CH₃COOH | 2,498 |
| KNO₃ | 300 |
| Mg(NO₃)₂•6H₂O | 25 |
| NaCH₃COO | 1,171 |
| NaNO₃ | 1,829 |
| Pb standard | 15 |

The concentrations in the table are those for the metal component, e.g., 25 ppm Ca²+, 300 ppm K⁺, etc. The total Na⁺ content was targeted at 3000 ppm coming from NaNO₃ and sodium acetate. The solution was buffered with acetic acid/sodium acetate buffer; acetic acid concentration was targeted at 2500 ppm while the acetic acid/sodium acetate ratio was targeted at 2.9. A standard 10,000 ppm Pb solution was used to bring the Pb concentration to a targeted value of 15 ppm Pb.

For the test, 0.1000 g solid titanate-based was mixed with 100 ml of the test solution giving a liquid/solid ratio L/S=1000. The uptake experiments were carried out in 125 ml HDPE bottles placed in a New Brunswick Innova 40 Incubator Orbital Shaker operating at 120 rpm at 25° C. for a period of 2-2.5 hr. After the exposure, 10 ml aliquots of the ion exchanged solutions as well as untreated starting feed were filtered through 0.2 μm Thermo Scientific™ Target 2™ Nylon/GMF filters. The elemental analysis of Pb was done using ICP-MS (Perkin Elmer NexION 300D) by taking 0.1000 ml of the solution and diluting them to 200 ml. Indium was added as the internal standard for the counts, while Sc and Bi were added as additional quality monitors. Two milliliters of nitric acid were added to the final solution as well. The detection level for Pb was 0.003 ppm or 3 ppb.

The K_{d} value for the distribution coefficient of metals between solution and solid was calculated using the following formula:

$${K}_{d} ( mL / g ) = \frac{( V ) ( Ac )}{( W ) ( Sc )} 1$$

where: V=volume of bodily fluid simulant (mL)
Ac=concentration of cation absorbed on ion-exchanger (g/mL)
W=mass of ion-exchanger evaluated (g)
Sc=concentration of cation in post reaction supernate (g/mL)

The Pb²⁺ uptake test results for the materials of each Example are given in Table 10, expressed in terms of K_{d}, the distribution coefficient. The uptake test results for Na⁺, K⁺, Mg²⁺ and Ca²⁺ are given in Table 10 B.

TABLE 10A

| Example | Pb²⁺ K_{d} (mL/g) |
|---|---|
| C1 | 258,700 |
| C2 | 504,300 |
| 1 | 856,600 |
| 2 | 296,100 |

TABLE 10B
Na⁺, K⁺, Mg²⁺, Ca²⁺ Uptake distribution coefficients, K_{d}, (mL/g)

| Example | Na⁺ | K⁺ | Mg²⁺ | Ca²⁺ |
|---|---|---|---|---|
| 1 | 13 | — | 29 | 291 |
| 2 | 23 | — | — | 398 |

The new materials of Examples 1 and 2 perform well for Pb²⁺ uptake, with the uniform Example 1 potassium titanate composition giving the best performance, better than that seen for the prior art alkali titanates of U.S. Pat. No. 11,964,266 ('266 patent). The Example 2 TiO₂ sphere-derived potassium titanate performs comparably to the sodium nonatitanate compound of the '266 patent. However, the Example C1 and C2 materials of the '266 patent do not meet the size standard to avoid substantial absorption in the gastrointestinal tract. The Example 1 and Example 2 materials are thus superior options for Pb²⁺ removal from the gastrointestinal tract. The results in Table 10B show that the alkali titanate ion exchangers of examples 1 and 2 have poor selectivity for Na⁺, K⁺, Mg²⁺ and Ca²⁺, the K_{d}s lower by 3-4 orders of magnitude. This is a favorable property of these alkali titanate ion exchangers that they don't upset the concentrations of other biologically important cations in bodily fluids providing only a minimally disruption.

What is claimed is:

1. An alkali titanate ion exchanger having an empirical formula on an anhydrous basis of: 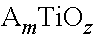
wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60,
wherein the alkali titanate ion exchanger is synthesized from a Ti source that is either nano-sized TiO₂ powder or preformed spray dried TiO₂ spheres, and wherein the alkali titanate ion exchanger exhibits a median particle size of greater than 3 microns (μm).

2. The ion exchanger of claim [0089], wherein the alkali titanate ion exchanger is an acid-treated alkali titanate ion exchanger.

3. The ion exchanger of claim [0089], wherein A is the potassium ion, or the hydronium ion, or a mixture thereof.

4. The ion exchanger of claim 1, wherein the alkali titanate ion exchanger has spherical morphology.

5. The ion exchanger of claim 1, wherein the alkali titanate ion exchanger has amorphous morphology.

6. The ion exchanger of claim 1, wherein the alkali titanate ion exchanger is a powder.

7. The ion exchanger of claim 1, wherein the median particle size is between 25 to 125 microns (μm).

8. The ion exchanger of claim 1, wherein less than 3% of the particles of the alkali titanate ion exchanger have a particle size of less than 3 microns (μm).

9. The ion exchanger of claim 1, wherein the alkali titanate ion exchanger has:
a particle size distribution d₁₀ value of between about 10 microns (μm) and about 80 μm; and
a particle size distribution d₅₀ value of between about 45 μm and about 125 μm; and
a particle size distribution d₉₀ value of between about 105 μm and about 190 μm.

10. The ion exchanger of claim 1, wherein the alkali titanate ion exchanger is
stable in a liquid environment at a pH of 1-2; or substantially insoluble at a pH range of 1-13; or both.

11. The ion exchanger of claim 1, wherein the alkali titanate ion exchanger has a distribution coefficient (K_{d}) for Pb²⁺ of between about 50,000 to about 5,500,000 milliliters per gram (mL/g) in solution.

12. A method for selectively removing Pb²⁺ toxins from gastrointestinal fluid, the process comprising contacting the fluid containing the toxins with an alkali titanate ion exchanger, resulting in an ion exchanged ion exchanger and thereby removing the Pb²⁺ toxins from the fluid, the alkali titanate ion exchanger having an empirical formula on an anhydrous basis of: 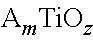
wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60,
wherein the alkali titanate ion exchanger is synthesized from a Ti source that is either nano-sized TiO₂ powder or preformed spray dried TiO₂ spheres, wherein the alkali titanate ion exchanger exhibits a median particle size of greater than 3 microns (μm), and wherein the alkali titanate ion exchanger minimally disrupts the levels of any one or more ions selected from Na⁺, Mg²⁺, K⁺, and Ca²⁺.

13. The method of claim 12, wherein the Pb²⁺ toxins are sequestered within the ion exchanged ion exchanger after the contacting.

14. An intracorporeal process for removing Pb²⁺ toxins from gastrointestinal fluid, the process comprising contacting the fluid containing the toxins with an alkali titanate ion exchanger resulting in an ion exchanged ion exchanger thereby removing the toxins from the fluid, the alkali titanate ion exchanger having an empirical formula on an anhydrous basis of: 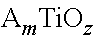
wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60, wherein the alkali titanate ion exchanger is synthesized from a Ti source that is either nano-sized TiO₂ powder or preformed spray dried TiO₂ spheres, and wherein the alkali titanate ion exchanger exhibits a median particle size of greater than 3 microns (μm).

15. A process for preparing an alkali titanate ion exchanger, the alkali titanate ion exchanger having an empirical formula on an anhydrous basis of: 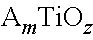
wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60, wherein the titanate-based ion exchanger is synthesized from titania (TiO₂) reagents, including nano-sized titania powder, preformed spray dried titania spheres, or both, the process comprising;
(a) forming a reaction mixture comprising reactive sources of A, Ti, and water; and
(b) heating the reaction mixture at a temperature of about 85° C. to about 225° C. for a time period of 0.5 to 30 days to form the alkali titanate ion exchanger;
wherein the reaction mixture comprises a composition expressed in terms of mole ratios of the oxides of: 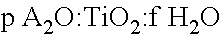
wherein “p” has a value from about 4 to 40; and “f” has a value from 20 to 1000.

16. The process of claim 15 wherein the Ti source is nano-sized TiO₂ powder, or preformed spray dried TiO₂ spheres.

17. The process of claim 16 wherein the nano-sized Ti source is selected from the group of nano-sized rutile titanium dioxide, nano-sized anatase titanium dioxide, nano-sized brookite titanium dioxide, nano-sized amorphous titanium dioxide, nano-sized titanium oxyhydroxide, and mixtures thereof, where the particle size is less than 200 nm, preferably 100 nm or less.

18. The process of claim 16 wherein the Ti source is preformed spray dried spheres.

19. The process of claim 18 wherein the Ti source for preformed spray dried TiO₂ spheres is amorphous TiO₂, amorphous titanium oxyhydroxide, anatase titanium dioxide, rutile titanium dioxide, brookite titanium dioxide, nano-sized rutile titanium dioxide, nano-sized anatase titanium dioxide, nano-sized brookite titanium dioxide, nano-sized amorphous titanium dioxide, nano-sized titanium oxyhydroxide, or mixtures thereof.

20. The process of claim 15 wherein the A source is selected from sodium hydroxide, potassium hydroxide, lithium hydroxide, or mixtures thereof.

21. A method for manufacturing a tablet or capsule or for oral administration, the tablet or capsule comprising an alkali titanate ion exchanger having an empirical formula on an anhydrous basis of: 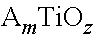
wherein
A is an exchangeable cation selected from the group consisting of potassium ion, sodium ion, lithium ion, calcium ion, magnesium ion, hydronium ion or mixtures thereof; “m” is the mole ratio of A to Ti and has a value from 0.10 to 0.60; and “z” is the mole ratio of O to Ti and has a value from 2.05 to 2.60, wherein the alkali titanate ion exchanger exhibits a median particle size of greater than 3 microns (μm),
the method comprising:
(a) forming a reaction mixture comprising reactive sources of A, Ti, and water,
(b) heating the reaction mixture for a period of time to form a alkali titanate ion exchanger;
(c) treating the synthesized alkali titanate ion exchanger via acid extraction and/or ion exchange with alkali metals, alkaline earth metals, or mixtures thereof, to form the alkali titanate ion exchanger with the desired composition;
(d) optionally admixing the alkali titanate ion exchanger with one or more pharmaceutically acceptable adjuvants, diluents or carriers to form an alkali titanate ion exchanger medicament;
(e) forming a capsule or tablet comprising the alkali titanate ion exchanger medicament;
wherein the reaction mixture comprises a composition expressed in terms of mole ratios of the oxides of: 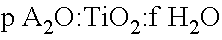
wherein “p” has a value from about 4 to 40; and “f” has a value from 20 to 1000.
